(19) United States
(12) Patent Application Publication
(10) Pub. No.: US 2026/0327194 A1
(43) Pub. Date: Oct. 22, 2026

(54) SYNTHESIZED THIAZOLIDINES AS A CYSTEINE DELIVERY METHOD IN CELL CULTURE FEED

(71) Applicant: Sanofi, Paris (FR)

(72) Inventors: Yuxin LIU, Hopkinton, MA (US); Yang Wu, Westborough, MA (US)

(21) Appl. No.: 19/684,738

(22) Filed: May 21, 2026

Related U.S. Application Data
(63) Continuation of application No. PCT/EP2024/083337, filed on Nov. 22, 2024.

(30) Foreign Application Priority Data
Nov. 24, 2023 (FR) 2313030
Mar. 18, 2024 (EP) 24305405.3

(51) Int. Cl. C07D 277/06; C12N 5/00

(52) U.S. Cl. CPC C07D 277/06; C12N 5/0018; C12N 2500/32

(57) ABSTRACT

The present disclosure, in some aspects, is directed to methods of making cysteine source solutions comprising a thiazolidine. In certain other aspects, the present disclosure is directed to methods of using the cysteine source solutions (such as in making a cell culture medium, performing cell culture, and/or producing a polypeptide), and systems and kits comprising the cysteine source solutions.

### CROSS-REFERENCE TO RELATED APPLICATIONS

This application is a continuation of International Application No. PCT/EP2024/083337, filed internationally on Nov. 22, 2024, which claims priority to and benefit of French Patent Application No. 2313030, filed on Nov. 24, 2023, and European Patent Application No. 24305405.3, filed on Mar. 18, 2024, the contents of each of which are hereby incorporated herein by reference in their entirety and for all purposes.

### TECHNICAL FIELD

The present disclosure, in some aspects, is directed to methods of making cysteine source solutions comprising a thiazolidine. In certain other aspects, the present disclosure is directed to methods of using the cysteine source solutions (such as in making a cell culture medium, performing cell culture, and/or producing a cell-product, such as a polypeptide), and systems and kits comprising the cysteine source solutions.

### BACKGROUND

For many cell types, cysteine is an important amino acid involved in, e.g., cell proliferation, metabolism, and stability, and has been linked to the cellular production of desirably high titers of recombinant proteins, e.g., antibodies. In culture, certain cell types need to be provided with exogenous cysteine. For example, in mammals, the liver regulates the free cysteine pool and thus in vivo there is a cysteine source for cells not capable of de novo cysteine synthesis. However, for certain mammalian cells cultured in isolation, a source of exogenous cysteine is required for sufficient cell growth and maintenance. Conventionally, exogenous cysteine is provided via cell culture media. However, cysteine has unfavorable properties when used in cell culture media as evidenced by the low solubility of cysteine at neutral pH. Due to this property, it is well known that cysteine often precipitates out of cell culture media, which greatly hinders the use of such cell culture media for adequately supporting cell growth and maintenance.

### BRIEF SUMMARY

In certain aspects, provided herein is a method of making a cysteine source solution suitable for use with a cell culture, the method comprising: reacting cysteine with an alpha-ketoacid compound of formula (A)

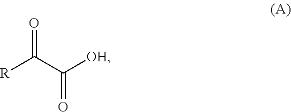

or the corresponding carboxylate anion, in an aqueous solution to produce the cysteine source solution comprising a thiazolidine compound of formula (B)

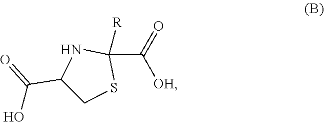

wherein R is optionally substituted C₁-C₆ alkyl, wherein the reaction is performed with: a concentration of the cysteine of at least about 30 mM, and/or a concentration of the alpha-ketoacid compound of formula (A), or corresponding carboxylate anion thereof, of at least about 30 mM, and/or at a pH of about 1.5 to about 12.5, and/or a ratio the cysteine and the alpha-ketoacid compound of formula (A) of about 2:1 to about 1:2.

In some embodiments, the reacting is not performed in a cell culture medium. In some embodiments, the reacting is performed for 5 hours or less.

In some embodiments, the residual concentration of cysteine or cystine in the cysteine source solution is about 0.5 mM or less. In some embodiments, the residual concentration of the alpha-ketoacid compound of formula (A) is about 0.5 mM or less.

In some embodiments, the purity of the thiazolidine compound of formula (B) in the resulting cysteine source solution is at least about 95% of the original cysteine concentration.

In some embodiments, the concentration of cysteine reacted with the alpha-ketoacid compound of formula (A) is about 30 mM to about 5000 mM. In some embodiments, the concentration of cysteine reacted with the alpha-ketoacid compound of formula (A) is at least about 150 mM. In some embodiments, the concentration of cysteine reacted with the alpha-ketoacid compound of formula (A) is at least about 1800 mM.

In some embodiments, the alpha-ketoacid compound of formula (A) is pyruvic acid (Pyr) or pyruvate. In some embodiments, the alpha-ketoacid compound of formula (A) is alpha-ketoglutaric acid (AKG) or alpha-ketoglutarate. In some embodiments, the alpha-ketoacid compound of formula (A) is oxaloacetic acid (Oxa) or oxaloacetate. In some embodiments, the concentration of the alpha-ketoacid compound of formula (A) reacted with cysteine is about 30 mM to about 5000 mM. In some embodiments, the concentration of the alpha-ketoacid compound of formula (A) reacted with cysteine is at least about 150 mM. In some embodiments, the concentration of the alpha-ketoacid compound of formula (A) reacted with cysteine is at least about 1800 mM.

In some embodiments, the reacting cysteine with the alpha-ketoacid compound of formula (A) is conducted at a pH from about 3 to about 12. In some embodiments, the reacting cysteine with the alpha-ketoacid compound of formula (A) is conducted at a pH of about 4.1. In some embodiments, the reacting cysteine with the alpha-ketoacid compound of formula (A) is conducted at a pH of about 7. In some embodiments, the reacting cysteine with the alpha-ketoacid compound of formula (A) is conducted at a pH of about 12.

In some embodiments, the cysteine and alpha-ketoacid compound of formula (A) are reacted at a concentration ratio from 1:1 to 1:2 cysteine: alpha-ketoacid. In some embodiments, the cysteine and alpha-ketoacid compound of formula (A) are reacted at a concentration ratio of 1:1 cysteine: alpha-ketoacid.

In some embodiments, cysteine is L-cysteine.

In other aspects, provided herein is a method of making a cysteine source solution suitable for use with a cell culture, the method comprising: reacting at least about 1800 mM cysteine with at least about 1800 mM of pyruvic acid, or the corresponding carboxylate anion, in an aqueous solution to produce the cysteine source solution comprising 2-methyl-1,3-thiazolidine-2,4-dicarboxylic acid, wherein the reaction is performed starting at a pH of about 3.5 to about 12. In some embodiments, the reaction is performed starting at a pH of about 4.1. In some embodiments, the reaction is performed starting at a pH of about 7.

In other aspects, provided herein is a method of making a cysteine source solution suitable for use with a cell culture, the method comprising: reacting at least about 1800 mM cysteine with at least about 1800 mM of alpha-ketoglutaric acid, or the corresponding carboxylate anion, in an aqueous solution to produce the cysteine source solution comprising 2-(2-carboxyethyl) thiazolidine-2,4-dicarboxylic acid, wherein the reaction is performed starting at a pH of about 4 to about 8. In some embodiments, the reaction is performed at a pH of about 7.

In other aspects, provided herein is a method of making a cysteine source solution suitable for use with a cell culture, the method comprising: reacting at least about 1800 mM cysteine with at least about 1800 mM of oxaloacetic acid, or the corresponding carboxylate anion, in an aqueous solution to produce the cysteine source solution comprising 2-(carboxymethyl) thiazolidine-2,4-dicarboxylic acid, wherein the reaction is performed starting at a pH of about 4 to about 8. In some embodiments, the reaction is performed at a pH of about 7.

In some embodiments, the reacting is performed for 2 hours or less. In some embodiments, the residual concentration of cysteine or cystine in the cysteine source solution is about 0.5 mM or less. In some embodiments, the residual concentration of pyruvic acid, alpha-ketoglutaric acid, or oxaloacetic acid, or the corresponding carboxylate anion of any, is about 0.5 mM or less. In some embodiments, the purity of the thiazolidine in the resulting cysteine source solution is at least about 95%.

In other aspects, provided herein is a cysteine source solution produced according to a method described herein.

In other aspects, provided herein is a method of making a cell culture medium, the method comprising admixing a basal cell culture medium with a cysteine source solution to make the cell culture medium, wherein the cysteine source solution is produced according to a method described herein. In some embodiments, the cell culture medium is suitable for cysteine-dependent cells. In some embodiments, the basal cell culture medium is not suitable for cysteine-dependent cells without further modification. In some embodiments, the basal cell culture medium has a pH of about 5 to about 8. In some embodiments, the method further comprises preparing the thiazolidine side solution according to a method described herein. In some embodiments, the method further comprises adjusting the pH of the basal cell culture medium. In some embodiments, the method further comprises QS the cell culture medium after admixing the basal cell culture medium and the cysteine side solution. In some embodiments, the basal cell culture medium is substantially free of cysteine and cystine.

In other aspects, provided herein is a method of culturing a cysteine-dependent cell, the method comprising culturing the cysteine-dependent cell in a cell culture medium made according a method described herein.

In other aspects, provided herein is a method of producing a polypeptide, the method comprising culturing a cysteine-dependent cell in a cell culture medium made according to a method described herein, and obtaining the polypeptide therefrom. In some embodiments, the polypeptide is a therapeutic polypeptide or a precursor thereof. In some embodiments, the therapeutic polypeptide or the precursor thereof is an antibody or a fragment thereof. In some embodiments, the antibody or a fragment thereof is an antibody drug conjugate.

In other aspects, provided herein is a cell culture system comprising: a cell; and a basal cell culture medium admixed with the cysteine source solution described herein, or a dried or semi-dried form thereof. In some embodiments, the basal cell culture medium is substantially free of cysteine and cystine.

In other aspects, provided herein is a method of culturing a cell, the method comprising providing a cell culture medium to the cell, wherein the cell culture medium comprises a basal culture medium and a cysteine source solution comprising a thiazolidine, and wherein and the cell culture medium does not comprise more than about 20% cysteine relative to the amount of the thiazolidine in the cell culture medium; and culturing the cell. In some embodiments, the cell is a cysteine-dependent cell. In some embodiments, the cell culture medium is not suitable for culturing the cell without further including cysteine or a source thereof. In some embodiments, the cysteine source solution is the sole source of cysteine for the cell. In some embodiments, the cell culture medium does not comprise cysteine. In some embodiments, the cysteine source solution is produced according to a method described herein.

### BRIEF DESCRIPTION OF THE DRAWINGS

FIG. 1A shows Cys/Pyr, Cys/AKG, and Cys/Oxa reaction profiles by DTNB assay in Optimization Test 1. FIG. 1B shows select DTNB assay data from FIG. 1A.

FIG. 2 shows Cys/Pyr, Cys/AKG, and Cys/Oxa reaction profiles by DTNB assay in Optimization Test 2.

FIG. 3A shows Cys/Pyr, Cys/AKG, and Cys/Oxa reaction profiles by DTNB assay in Optimization Test 3. FIG. 3B shows select DTNB assay data from FIG. 3A.

FIG. 4A shows representative chromatograms obtained by applying the methods described herein. FIG. 4B shows representative chromatograms obtained by applying the methods described herein.

FIGS. 5A-5C shows chemical structures of three complexes (A) Cys-Pyr, (B) Cys-AKG, and (C) Cys-Oxa.

FIG. 6 shows Cys-Pyr molecule corresponding to peak RT-6.04, with theoretical fragment weights and experimental data.

FIGS. 7A and 7B show MS-MS of Cys-Pyr molecule fragments and daughter ions with fragment structures predicted by Mass Frontier™ (Version 8.0 SR1).

FIG. 8 shows Cys-AKG molecule corresponding to peak RT=11.18, with theoretical fragment weights and experimental data.

FIGS. 9A and 9B show MS-MS of Cys-AKG molecule fragments and daughter ions with fragment structures predicted by Mass Frontier™ (Version 8.0 SR1).

FIG. 10 shows Cys-Oxa molecule corresponding to peak RT=9.20 with theoretical fragment weights and experimental data.

FIGS. 11A and 11B show MS-MS of Cys-Oxa molecule fragments and daughter ions with fragment structures predicted by Mass Frontier™ (Version 8.0 SR1).

FIG. 12 shows VCD profiles of four different cell lines in ambr15 fed-batch bioreactor with six Cys delivery methods.

FIG. 13 shows viability profiles of four different cell lines in ambr15 fed-batch bioreactor with six Cys delivery methods.

FIG. 14 shows titer profiles of four different cell lines in ambr15 fed-batch bioreactor with six Cys delivery methods.

FIGS. 15A-15F show plots of normalized free cysteine concentration over time for certain cell culture media conditions.

FIGS. 16A-16E show plots indicative of cell culture perform and product quality for certain cell cultures.

FIGS. 17A-17B show plots of certain metabolites studied over time for certain cell culture media conditions.

### DETAILED DESCRIPTION

Provided in the present application, in certain aspects, are methods of making cysteine source solutions suitable for providing a cysteine source for cell culture, wherein the cysteine source solution comprises a thiazolidine compound. As described herein, cysteine sources solutions are useful for replacing the need to provide cysteine directly to a cell culture medium. Thus, in some embodiments, the cysteine source solution does not comprise cysteine (or does not comprise a substantial amount of cysteine, such as does not comprise more than about 20%, such as 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less, relative to the thiazolidine amount). The disclosure of the present application is based, at least in part, on the inventors' findings regarding efficient methods for producing cysteine source solutions having a high yield and purity of thiazolidine compounds suitable to replace other cysteine sources conventionally used in cell culture. Said synthesized thiazolidine compounds were found to be highly soluble and stable in the described cysteine source solutions and cell culture media, which overcomes a significant disadvantage of using cysteine directly, namely, known solubility issues of cysteine in cell culture media leading cysteine precipitation. Precipitated cysteine is not usable by cells and furthermore presents manufacturing quality control issues. Moreover, it was found that cells are able to use the synthesized thiazolidines as a cysteine source. As demonstrated in the Examples, the cysteine source solutions taught herein can be combined with basal cell culture media lacking other cysteine sources and the resulting cell culture media provide an environment suitable for cell maintenance, growth, and production of desired titers of recombinant polypeptides. Moreover, the thiazolidines of the taught cysteine source solutions (or compositions comprising the same such as cell culture media) are highly soluble do not lead to the presence of precipitated cysteine. Such thiazolidines of the taught cysteine source solutions have a high bioavailability and are non-toxic, can be readily produced, have a high solubility, are suitable for single feed cell culture techniques, protect the thiol group on the cysteine component from redox reaction, and are a cost-effective approach for providing a cysteine source for cell culture. The advantages provided by the cysteine source solutions described herein, including the advantages of the methods for producing said cysteine source solutions, represent a significant advancement in the field of cell culture and the products that can produced therefrom.

Thus, in some aspects, provided herein is method of making a cysteine source solution suitable for use with a cell culture, the method comprising reacting cysteine with an alpha-ketoacid compound in an aqueous solution to produce the cysteine source solution comprising a thiazolidine compound, wherein the reaction is performed with: a concentration of the cysteine of at least about 30 mM, and/or a concentration of the alpha-ketoacid compound of formula (A), or corresponding carboxylate anion thereof, of at least about 30 mM, and/or at a pH of about 1.5 to about 12.5, and/or a ratio the cysteine and the alpha-ketoacid compound of formula (A) of about 2:1 to about 1:2.

In other aspects, provided herein is a method of making a cysteine source solution suitable for use with a cell culture, the method comprising: reacting cysteine with an alpha-ketoacid compound of formula (A)

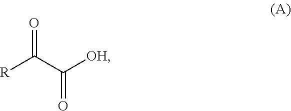

or the corresponding carboxylate anion, in an aqueous solution to produce the cysteine source solution comprising a thiazolidine compound of formula (B)

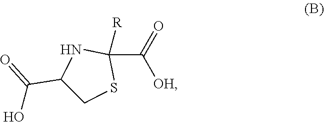

wherein R is optionally substituted C₁-C₆ alkyl,
wherein the reaction is performed with:
   a concentration of the cysteine of at least about 30 mM, and/or
   a concentration of the alpha-ketoacid compound of formula (A), or corresponding carboxylate anion thereof, of at least about 30 mM, and/or
   at a pH of about 1.5 to about 12.5, and/or
   a ratio the cysteine and the alpha-ketoacid compound of formula (A) of about 2:1 to about 1:2.

In other aspects, provided is a method of making a cysteine source solution suitable for use with a cell culture, the method comprising: reacting at least about 1800 mM, including about 1800 mM, cysteine with at least about 1800 mM, including about 1800 mM, of pyruvic acid, or the corresponding carboxylate anion, in an aqueous solution to produce the cysteine source solution comprising 2-methyl-1,3-thiazolidine-2,4-dicarboxylic acid, wherein the reaction is performed starting at a pH of about 3.5 to about 12. In some embodiments, the reaction is performed starting at a pH of about 4.1. In some embodiments, the reaction is performed starting at a pH of about 7.

In other aspects, provided is a method of making a cysteine source solution suitable for use with a cell culture, the method comprising: reacting at least about 1800 mM, including about 1800 mM, cysteine with at least about 1800 mM, including about 1800 mM, of alpha-ketoglutaric acid, or the corresponding carboxylate anion, in an aqueous solution to produce the cysteine source solution comprising 2-(2-carboxyethyl) thiazolidine-2,4-dicarboxylic acid, wherein the reaction is performed starting at a pH of about 4 to about 8. In some embodiments, the reaction is performed at a pH of about 7.

In other aspects, provided is a method of making a cysteine source solution suitable for use with a cell culture, the method comprising: reacting at least about 1800 mM, including about 1800 mM, cysteine with at least about 1800 mM, including about 1800 mM, of oxaloacetic acid, or the corresponding carboxylate anion, in an aqueous solution to produce the cysteine source solution comprising 2-(carboxymethyl) thiazolidine-2,4-dicarboxylic acid, wherein the reaction is performed starting at a pH of about 4 to about 8. In some embodiments, the reaction is performed at a pH of about 7.

In other aspects, provided is a cysteine source solution produced according to any method described herein.

In other aspects, provided is a method of making a cell culture medium, the method comprising admixing a basal cell culture medium with a cysteine source solution to make the cell culture medium, wherein the cysteine source solution is produced according to any method described herein.

In other aspects, provided is a method of culturing a cysteine-dependent cell, the method comprising culturing the cysteine-dependent cell in a cell culture medium made according to the description provided herein.

In other aspects, provided is a method of producing a polypeptide, the method comprising culturing a cysteine-dependent cell in a cell culture medium made according to the description provided herein, and obtaining the polypeptide therefrom.

In other aspects, provided is a cell culture system comprising: a cell; and a basal cell culture medium admixed with a cysteine source solution described herein.

### A. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they comprise a Fc domain.

The terms “full length antibody,” is used herein to refer to an antibody having a structure substantially similar to a native antibody structure or having heavy chains that contain an Fc region as defined herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “chimeric” antibody refers to an antibody in which a portion of the heavy and/or light chain is derived from a particular source or species, while the remainder of the heavy and/or light chain is derived from a different source or species.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); an immunoglobulin single variable domain, such as a VHH; and multispecific antibodies formed from antibody fragments.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method.

The term “therapeutic antibody” refers to an antibody that is used in the treatment of disease. A therapeutic antibody may have various mechanisms of action. A therapeutic antibody may bind and neutralize the normal function of a target associated with an antigen. For example, a monoclonal antibody that blocks the activity of the of protein needed for the survival of a cancer cell causes the cell's death. Another therapeutic monoclonal antibody may bind and activate the normal function of a target associated with an antigen. For example, a monoclonal antibody can bind to a protein on a cell and trigger an apoptosis signal. Yet another monoclonal antibody may bind to a target antigen expressed only on diseased tissue; conjugation of a toxic payload (effective agent), such as a chemotherapeutic or radioactive agent, to the monoclonal antibody can create an agent for specific delivery of the toxic payload to the diseased tissue, reducing harm to healthy tissue. A “biologically functional fragment” of a therapeutic antibody will exhibit at least one if not some or all of the biological functions attributed to the intact antibody, the function comprising at least specific binding to the target antigen. In some embodiments, the therapeutic antibody is an antibody conjugate, e.g., an antibody drug conjugate. In some embodiments, the feature conjugated to the antibody is done so covalently, including through a linker, or non-covalently.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open-ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

Those skilled in the art will recognize that several embodiments are possible within the scope and spirit of the present disclosure. The following description illustrates the disclosure and, of course, should not be construed in any way as limiting the scope of the inventions described herein.

### B. Methods of Making Cysteine Source Solutions

Provided herein are methods of making a cysteine source solution suitable for use with a cell culture and methods of using the same. In some embodiments, the methods of making a cysteine source solution comprising reacting cysteine with an alpha-ketoacid compound to produce a cysteine source solution comprising a thiazolidine compound. As described herein, in certain embodiments, the reaction is performed with: a concentration of the cysteine of at least about 30 mM, and/or a concentration of the alpha-ketoacid compound, or corresponding carboxylate anion thereof, of at least about 30 mM, and/or, at a pH of about 1.5 to about 12.5, and/or a ratio the cysteine and the alpha-ketoacid compound of about 2:1 to about 1:2.

In some embodiments, provided herein is a method of making and/or using a cysteine source solution suitable for use with a cell culture. In some such embodiments, the method comprises: reacting cysteine with an alpha-ketoacid compound of formula (A):

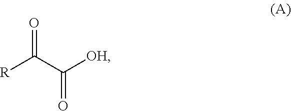

or the corresponding carboxylate anion compound of formula (A-I):

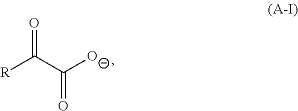

in an aqueous solution to produce the cysteine source solution comprising a thiazolidine compound of formula (B):

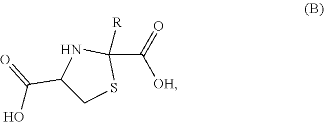

wherein R is optionally substituted aliphatic, optionally substituted unsaturated alkyl, C(O)R¹, C(O)OR², or —OR¹;
wherein R¹ is alkyl optionally substituted by R² or C(O)R²; and
R² is optionally substituted alkyl or H;
wherein the reaction is performed with:
   a concentration of the cysteine of at least about 30 mM, and/or
   a concentration of the alpha-ketoacid compound of formula (A), or corresponding carboxylate anion compound of formula (A-I), of at least about 30 mM, and/or
   at a pH of about 1.5 to about 12.5, and/or
   a ratio of the cysteine to the alpha-ketoacid compound of formula (A), or corresponding carboxylate anion compound of formula (A-I), of about 4:1 to about 1:4.

Certain embodiments provided herein describe a pH of a reaction. It is to be understood that such pH is the pH at the start of performing the reaction, e.g., mixing two or more reactants, unless noted otherwise. Such teaching is provided in appreciation that the pH of the reaction mixture can change over the course of a reaction. In some embodiments, the pH of a reaction will change during the course to a pH outside a stated range for a reaction, and such embodiments are not outside of the teachings of the present description unless the specifically noted in such embodiment provided herein.

As disclosed herein, aspects of the methods provided herein may be performed in a number of ways and formats. Further discussion of aspects of the methods provided is included in the sections below. The modular discussion of such aspects does not limit the scope of the invention and one of ordinary skill in the art will readily appreciate how certain features from the sections below and herein can be combined to perform the methods taught herein.

### i. Cysteines

The methods of making a cysteine source solution suitable for use with a cell culture provided herein comprise a cysteine as a reactant. As described herein, the cysteine may come in various forms and the disclosure envisions such forms are readily substitutable (and may be present in certain mixtures) unless taught otherwise.

In some embodiments, the cysteine is L-cysteine.

In some embodiments, the cysteine is present with an acid, e.g., cysteine hydrochloric acid. In some embodiments, the cysteine is hydrated, e.g., a monohydrate. In some embodiments, the cysteine is formulated as an ester, e.g., cysteine methyl ester.

In some embodiments, the cysteine is in the form of a solution. In some embodiments the cysteine is in the form of a powder, such as a lyophilized powder.

In some embodiments, the reactions described herein are performed with a concentration of the cysteine of about 30 mM to about 5000 mM, such as any of about 500 mM to about 2000 mM, about 500 mM to about 1250 mM, about 750 mM to about 1500 mM, about 1000 mM to about 2000 mM, about 1500 mM to about 2000 mM, about 1700 mM to about 1900 mM, about 1500 mM to about 5000 mM, or about 2500 mM to about 5000 mM. In some embodiments, the reactions described herein are performed with a concentration of the cysteine of at least about 30 mM, such as at least about any of 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1050 mM, 1100 mM, 1150 mM, 1200 mM, 1250 mM, 1300 mM, 1350 mM, 1400 mM, 1450 mM, 1500 mM, 1550 mM, 1600 mM, 1650 mM, 1700 mM, 1750 mM, 1800 mM, 1850 mM, 1900 mM, 1950 mM, 2000 mM, 2050 mM, 2100 mM, 2150 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 mM, 4100 mM, 4200 mM, 4300 mM, 4400 mM, 4500 mM, 4600 mM, 4700 mM, 4800 mM, 4900 mM, or 5000 mM. In some embodiments, the reactions described herein are performed with a concentration of the cysteine of about any of 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1050 mM, 1100 mM, 1150 mM, 1200 mM, 1250 mM, 1300 mM, 1350 mM, 1400 mM, 1450 mM, 1500 mM, 1550 mM, 1600 mM, 1650 mM, 1700 mM, 1750 mM, 1800 mM, 1850 mM, 1900 mM, 1950 mM, 2000 mM, 2050 mM, 2100 mM, 2150 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 mM, 4100 mM, 4200 mM, 4300 mM, 4400 mM, 4500 mM, 4600 mM, 4700 mM, 4800 mM, 4900 mM, or 5000 mM. One of ordinary skill in the art will readily appreciate the form (including workable variations thereof) of the cysteine reactant necessary to achieve the desired cysteine concentration for the reactions described herein. Moreover, said amount of cysteine indicate the starting amount of cysteine present at the start of the reaction with the alpha-ketoacid, and it is well understood that such initial reactants will decrease in a successful reaction.

### ii. Alpha-Ketoacids

The methods of making a cysteine source solution suitable for use with a cell culture provided herein comprise an alpha-ketoacid, or the corresponding carboxylate anion, as a reactant. As described herein, the alpha-ketoacid, or the corresponding carboxylate anion, may come in various forms and the disclosure envisions such forms are readily substitutable (and may be present in certain mixtures) unless taught otherwise.

In some embodiments, the alpha-ketoacid is a compound of formula (A)

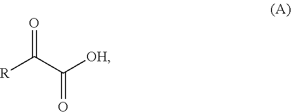

wherein R is optionally substituted aliphatic, optionally substituted unsaturated alkyl, C(O)R¹, C(O)OR², or —OR¹; wherein R¹ is alkyl optionally substituted by R² or C(O)R²; and R² is optionally substituted alkyl or H. In some such embodiments, R is optionally substituted 1- to 6-membered aliphatic. In some such embodiments, R is optionally substituted C₁-C₆ alkyl. In some such embodiments, R is C₁-C₆ alkyl optionally substituted with halo, OH, SH, 6- to 10-membered aryl, or C(O)OR², wherein the 6- to 10-membered aryl is optionally substituted with one or more groups selected from OH, C₁-C₆ alkyl, or C(O)OR². In some such embodiments, R is C₁-C₆ alkyl optionally substituted with one or more C(O)OR², wherein R² is H or C₁-C₆ alkyl. In some such embodiments, R is C₁-C₆ alkyl optionally substituted with COOH. In some such embodiments, R is C₁-C₆ alkyl substituted with one or more COOH. In some such embodiments, R is C₂-C₆ alkenyl optionally substituted with one or more groups selected from halo, OH, SH, or C(O)OR². In some such embodiments, R is C(O)R¹, wherein R¹ is C₁-C₆ alkyl. In some such embodiments, R is C(O)OR², wherein R² is C₁-C₆ alkyl or H. In some such embodiments, R is —OR¹, wherein R¹ is C₁-C₆ alkyl optionally substituted by R². In some such embodiments, the alpha-ketoacid is the corresponding carboxylate anion of formula (A-I)

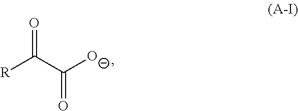

wherein the variable group R is as previously defined for formula (A).

In some embodiments, the alpha-ketoacid of formula (A) is pyruvic acid, alpha-ketoglutaric acid, or oxaloacetic acid or the corresponding carboxylate anion of formula (A-I) is pyruvate, alpha-ketoglutarate, or oxaloacetate. In some such embodiments, the alpha-ketoacid is pyruvic acid. In some such embodiments, the corresponding carboxylate anion is pyruvate. In some such embodiments, the alpha-ketoacid is alpha-ketoglutaric acid. In some such embodiments, the corresponding carboxylate anion is alpha-ketoglutarate. In some such embodiments, the alpha-ketoacid is oxaloacetic acid. In some such embodiments, the corresponding carboxylate anion is oxaloacetate.

In some embodiments, the reactions described herein are performed with a concentration of the alpha-ketoacid, or the corresponding carboxylate anion (e.g., pyruvic acid, alpha-ketoglutaric acid, or oxaloacetic acid, or the corresponding carboxylate anion of any), of about 30 mM to about 5000 mM, such as any of about 500 mM to about 2000 mM, about 500 mM to about 1250 mM, about 750 mM to about 1500 mM, about 1000 mM to about 2000 mM, about 1500 mM to about 2000 mM, about 1700 mM to about 1900 mM, about 1500 mM to about 5000 mM, or about 2500 mM to about 5000 mM. In some embodiments, the reactions described herein are performed with a concentration of the alpha-ketoacid, or the corresponding carboxylate anion (e.g., pyruvic acid, alpha-ketoglutaric acid, or oxaloacetic acid, or the corresponding carboxylate anion of any), of at least about 30 mM, such as at least about any of 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1050 mM, 1100 mM, 1150 mM, 1200 mM, 1250 mM, 1300 mM, 1350 mM, 1400 mM, 1450 mM, 1500 mM, 1550 mM, 1600 mM, 1650 mM, 1700 mM, 1750 mM, 1800 mM, 1850 mM, 1900 mM, 1950 mM, 2000 mM, 2050 mM, 2100 mM, 2150 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 mM, 4100 mM, 4200 mM, 4300 mM, 4400 mM, 4500 mM, 4600 mM, 4700 mM, 4800 mM, 4900 mM, or 5000 mM. In some embodiments, the reactions described herein are performed with a concentration of the alpha-ketoacid, or the corresponding carboxylate anion (e.g., pyruvic acid, alpha-ketoglutaric acid, or oxaloacetic acid, or the corresponding carboxylate anion of any), of about any of 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1050 mM, 1100 mM, 1150 mM, 1200 mM, 1250 mM, 1300 mM, 1350 mM, 1400 mM, 1450 mM, 1500 mM, 1550 mM, 1600 mM, 1650 mM, 1700 mM, 1750 mM, 1800 mM, 1850 mM, 1900 mM, 1950 mM, 2000 mM, 2050 mM, 2100 mM, 2150 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 mM, 4100 mM, 4200 mM, 4300 mM, 4400 mM, 4500 mM, 4600 mM, 4700 mM, 4800 mM, 4900 mM, or 5000 mM. One of ordinary skill in the art will readily appreciate the form (including workable variations thereof) of the alpha-ketoacid reactant, or the corresponding carboxylate anion (e.g., pyruvic acid, alpha-ketoglutaric acid, or oxaloacetic acid, or the corresponding carboxylate anion of any), necessary to achieve the desired the alpha-ketoacid, or the corresponding carboxylate anion, concentration for the reactions described herein. Moreover, said amount of the alpha-ketoacid, or the corresponding carboxylate anion (e.g., pyruvic acid, alpha-ketoglutaric acid, or oxaloacetic acid, or the corresponding carboxylate anion of any), indicate the starting amount of the alpha-ketoacid, or the corresponding carboxylate anion, present at the start of the reaction with the cysteine, and it is well understood that such initial reactants will decrease in a successful reaction.

### iii. Ratios of Reactants

The methods of making a cysteine source solution suitable for use with a cell culture provided herein encompass embodiments based on, at least in part, a ratio between reactants, namely, a cysteine and an alpha-ketoacid, or the corresponding carboxylate anion. In some embodiments, the reaction of a cysteine and an alpha-ketoacid, or the corresponding carboxylate anion, proceeds in a one-to-one manner to produce a thiazolidine. In some embodiments, the reaction described herein is configured based on a desired composition of a cysteine source solution following completion of a reaction described herein. For example, in some embodiments, the cysteine source solution comprises substantially no, including no, cysteine following the reaction, and in such reaction the ration of the cysteine and an alpha-ketoacid, or the corresponding carboxylate anion, is 1:1. In other embodiments, it may be desirable to have a remaining amount of a cysteine and/or an alpha-ketoacid, or the corresponding carboxylate anion, in the cysteine source solution following the completion of a reaction described herein. In such embodiments, the ratio of reactants can be established accordingly.

In some embodiments, the molar ratio of the cysteine and the alpha-ketoacid, or the corresponding carboxylate anion, at the start of a reaction described herein is about 4:1 to about 1:4, such as about any of about 3:1 to about 1:3, about 2.5:1 to about 1:2.5, about 2:1 to about 1:2, or about 1.5:1 to about 1:1.5. In some embodiments, the molar ratio of the cysteine and the alpha-ketoacid, or the corresponding carboxylate anion, at the start of a reaction described herein is about any of 4:1, 3.5:1, 3:1, 2.5:1, 2:1, 1.5:1, 1:1, 1:1.5, 1:2, 1:2.5, 1:3, 1:3.5, or 1:4. In some embodiments, the molar ratio of the cysteine and the alpha-ketoacid, or the corresponding carboxylate anion, at the start of a reaction described herein is about 1:1.

### iv. Reaction Conditions and Characteristics

The methods of making a cysteine source solution provided herein can be performed at various conditions.

As described herein, the reactions described herein can exhibits changes in pH as the reaction progresses, and thus the description of a pH for the reaction, including a starting pH, describes when the reactants are brought together for the reacting step. In some embodiments, the pH as the reaction progresses, such as during and following completion of thiazolidine production, is outside of the stated pH for the reaction. Such embodiments are still encompassed by the instant application. In some embodiments, the reaction is performed at a pH, including starting pH, of about 1 to about 13, such as any of about 1.5 to about 12.5, about 3 to about 12, about 3 to about 5, about 6 to about 8, or about 11 to about 13. In some embodiments, the reaction is performed at a pH, including starting pH, of at least about 3, such as at least about any of 4, 5, 6, 7, 8, 9, 10, 11, or 12. In some embodiments, the reaction is performed at a pH, including starting pH, of about 12 or less, such as about any of 11 or less, 10 or less, 9 or less, 8 or less, 7 or less, 6 or less, 5 or less, 4 or less, 3 or less, or 2 or less. In some embodiments, the reaction is performed at a pH, including starting pH, of about any of 3, 3.5, 3.6, 3.7, 3.8, 3.9, 4, 4.1, 4.2, 4.3, 4.4, 4.5, 4.6, 5, 6, 6.5, 6.6, 6.7, 6.8, 6.9, 7, 7.1, 7.2, 7.3, 7.4, 7.5, 8, 9, 9, 10, 11, 11.5, 11.6, 11.7, 11.8, 11.9, 12, 12.1, 12.2, 12.3, 12.4, or 12.5. In some embodiments, the reaction is performed at a pH, including starting pH, of about 4.1. In some embodiments, the reaction is performed at a pH, including starting pH, of about 7. In some embodiments, the reaction is performed at a pH, including starting pH, of about 12.

In some embodiments, the reacting is performed for about 5 hours or less, such as about any of 4.5 hours or less, 4 hours or less, 3.5 hours or less, 3 hours or less, 2.5 hours or less, 2 hours or less, 1.5 hours or less, or 1 hour or less.

The reactions taught herein can be performed at various other conditions. For examples, in some embodiments, the reacting is performed at about 10° C. to about 50° C., including room temperature or 37° C. In some embodiments, the reacting is performed in light. In some embodiments, the reacting is performed in dark. In some embodiments, the reacting is performed under inert atmosphere conditions.

In some embodiments, the reacting in not performed in a cell culture medium.

### v. Forms of the Resulting Cysteine Source Solutions

The reactions described herein provide cysteine source solutions suitable for use with a cell culture. The cysteine source solutions provided herein may come in a diverse array of forms, such as with varying concentrations of a thiazolidine, and, in some embodiments, can be further modified. In some embodiments, the cysteine source solution does not comprise cysteine (or does not comprise a substantial amount of cysteine, such as does not comprise more than about 2% relative to the thiazolidine amount, such as 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less). As discussed herein, in some embodiments, the cysteine source solution can be in a dried or semi-dried form.

In some embodiments, the cysteine source solution comprises a residual concentration of cysteine or cystine (including the sum thereof) in the cysteine source solution is about 1 mM or less, such as about any of 0.95 mM or less, 0.9 mM or less, 0.85 mM or less, 0.8 mM or less, 0.75 mM or less, 0.7 mM or less, 0.65, mM or less, 0.6 mM or less, 0.55 mM or less, 0.5 mM or less, 0.45 mM or less, 0.4 mM or less, 0.35 mM or less, 0.3 mM or less, 0.25 mM or less, 0.2 mM or less, 0.15 mM or less, 0.1 mM or less, or 0.05 mM or less. In some embodiments, the cysteine source solution comprises an amount of residual cysteine, including absence or substantially no, such that the resulting cysteine source solution does not form (or does not substantially form) cysteine precipitates (such as compared to a solution comprising an equivalent amount of cysteine as based on the amount of a thiazolidine in a cysteine source solution or a composition comprising the same).

In some embodiments, the cysteine source solution comprises a residual concentration of the alpha-ketoacid, or the corresponding carboxylate anion (including the sum thereof), in the cysteine source solution is about 1 mM or less, such as about any of 0.95 mM or less, 0.9 mM or less, 0.85 mM or less, 0.8 mM or less, 0.75 mM or less, 0.7 mM or less, 0.65, mM or less, 0.6 mM or less, 0.55 mM or less, 0.5 mM or less, 0.45 mM or less, 0.4 mM or less, 0.35 mM or less, 0.3 mM or less, 0.25 mM or less, 0.2 mM or less, 0.15 mM or less, 0.1 mM or less, or 0.05 mM or less.

In some embodiments, the yield of a thiazolidine in a cysteine source solution is at least about 90% (as compared to the expected amount of thiazolidine from a complete reaction or reactants), such at least about any of 91%, 92%, 93%, 94%, 95%, 96%, 97%, 98%, 99%, or 100%. In some embodiments, the concentration of a thiazolidine produced by the reaction is at least about 90% of the concentration of the limiting reagent(s), such as at least about any of 91%, 92%, 93%, 94%, 95%, 96%, 97%, 98%, 99%, or 100%. In some embodiments, the purity of a thiazolidine compound in the resulting cysteine source solution is at least about 90% relative to any residual cysteine or alpha-ketoacid, of the corresponding carboxylate anion, such as at least about any of 91%, 92%, 93%, 94%, 95%, 96%, 97%, 98%, 99%, or 100%.

In some embodiments, the cysteine source solution comprises a concentration of the thiazolidine of about 30 mM to about 5000 mM, such as any of about 500 mM to about 2000 mM, about 500 mM to about 1250 mM, about 750 mM to about 1500 mM, about 1000 mM to about 2000 mM, about 1500 mM to about 2000 mM, about 1700 mM to about 1900 mM, about 1500 mM to about 5000 mM, or about 2500 mM to about 5000 mM. In some embodiments, the cysteine source solution comprises a concentration of the thiazolidine of at least about 30 mM, such as at least about any of 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1050 mM, 1100 mM, 1150 mM, 1200 mM, 1250 mM, 1300 mM, 1350 mM, 1400 mM, 1450 mM, 1500 mM, 1550 mM, 1600 mM, 1650 mM, 1700 mM, 1750 mM, 1800 mM, 1850 mM, 1900 mM, 1950 mM, 2000 mM, 2050 mM, 2100 mM, 2150 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 mM, 4100 mM, 4200 mM, 4300 mM, 4400 mM, 4500 mM, 4600 mM, 4700 mM, 4800 mM, 4900 mM, or 5000 mM. In some embodiments, the cysteine source solution comprises a concentration of the thiazolidine of about any of 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1050 mM, 1100 mM, 1150 mM, 1200 mM, 1250 mM, 1300 mM, 1350 mM, 1400 mM, 1450 mM, 1500 mM, 1550 mM, 1600 mM, 1650 mM, 1700 mM, 1750 mM, 1800 mM, 1850 mM, 1900 mM, 1950 mM, 2000 mM, 2050 mM, 2100 mM, 2150 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 mM, 4100 mM, 4200 mM, 4300 mM, 4400 mM, 4500 mM, 4600 mM, 4700 mM, 4800 mM, 4900 mM, or 5000 mM.

In some embodiments, the cysteine source solution is a liquid, such as directly obtained from a reaction described herein. In some embodiments, the cysteine source solution is quantum satised (QS′ed) to a final desired volume and/or measurement. In some embodiments, the cysteine source solution comprises water and a thiazolidine.

In some embodiments, the cysteine source solution may be, following the completion of the reaction, processed into a dried form, such as via lyophilization, crystallization, and/or another technique for drying. For purposes of brevity, embodiments herein refer to cysteine source solutions, however, one of ordinary skill in the art will readily appreciate when cysteine source solution can be a derivative thereof, such as a dried form of a cysteine source solution. For example, in some embodiments, it is provided that a cysteine source solution is admixed with a basal culture medium. In some embodiments, the cysteine source solution is in liquid form and admixed with the basal culture medium. In some embodiments, the cysteine source solution is in a dried or semi-dried form and admixed with the basal culture medium.

As described herein, the provided cysteine source solutions are suitable for use with cell culture. One of ordinary skill in the art will readily appreciate the cysteine source solutions encompassed by such description, including how to assess suitability and that such description does not mean that the cysteine source solutions are suitable for all cell culture. In some embodiments, the suitability for use with a cell culture is based on suitability for a single cell type. Additional description of cell culturing is provided in other sections herein, and is a skill well-known in the art.

### vi. Example Embodiments

Provided herein, in certain aspects, is a method of making a cysteine source solution suitable for use with a cell culture, the method comprising: reacting about 1000 mM to about 2500 mM cysteine with about 1000 mM to about 2500 mM pyruvic acid, or the corresponding carboxylate anion, in an aqueous solution to produce the cysteine source solution comprising 2-methyl-1,3-thiazolidine-2,4-dicarboxylic acid, wherein the reaction is performed starting at a pH of about 3.5 to about 12. In some embodiments, the reaction comprises reacting at least about 1500 mM cysteine, such as at least about any of at 1600 mM cysteine, 1700 mM cysteine, 1800 mM cysteine, 1900 mM cysteine, or 2000 mM cysteine. In some embodiments, the reaction comprises reacting about 1800 mM cysteine. In some embodiments, the reaction comprises reacting at least about 1500 mM pyruvic acid, or the corresponding carboxylate anion, such as at least about any of at 1600 mM pyruvic acid, or the corresponding carboxylate anion, 1700 mM pyruvic acid, or the corresponding carboxylate anion, 1800 mM pyruvic acid, or the corresponding carboxylate anion, 1900 mM pyruvic acid, or the corresponding carboxylate anion, or 2000 mM pyruvic acid, or the corresponding carboxylate anion. In some embodiments, the reaction comprises reacting about 1800 mM pyruvic acid, or the corresponding carboxylate anion. In some embodiments, the reaction is performed starting at a pH of about 3.5 to about 4.5, including about 4.1. In some embodiments, the reaction is performed starting at a pH of about 6.5 to about 7.5, including about 7. As described herein, the reactions described herein can exhibits changes in pH as the reaction progresses, and thus the description of a starting pH describes when the reactants are brought together for the reacting step. In some embodiments, the reaction comprises reacting cysteine and pyruvic acid, or the corresponding carboxylate anion, at a ratio of about 2:1 to about 1:2, including a ratio of about 1:1. In some embodiments, the reacting is performed for about 5 hours or less, including about any of 4 hours or less, 3 hours or less, 2 hours or less, or 1 hour or less. In some embodiments, the residual concentration of cysteine or cystine in the cysteine source solution is about 0.5 mM or less. In some embodiments, the residual concentration of pyruvic acid, or the corresponding carboxylate anion of any, is about 0.5 mM or less. In some embodiments, the purity of the thiazolidine in the resulting cysteine source solution is at least about 95%, such as at least about any of 96%, 97%, 98%, 99%, or 100%. In some embodiments, the cysteine is L-cysteine.

Provided herein, in certain aspects, is a method of making a cysteine source solution suitable for use with a cell culture, the method comprising: reacting about 1000 mM to about 2500 mM cysteine with about 1000 mM to about 2500 mM alpha-ketoglutaric acid, or the corresponding carboxylate anion, in an aqueous solution to produce the cysteine source solution comprising 2-(2-carboxyethyl) thiazolidine-2,4-dicarboxylic acid, wherein the reaction is performed starting at a pH of about 4 to about 8. In some embodiments, the reaction comprises reacting at least about 1500 mM cysteine, such as at least about any of at 1600 mM cysteine, 1700 mM cysteine, 1800 mM cysteine, 1900 mM cysteine, or 2000 mM cysteine. In some embodiments, the reaction comprises reacting about 1800 mM cysteine. In some embodiments, the reaction comprises reacting at least about 1500 mM alpha-ketoglutaric acid, or the corresponding carboxylate anion, such as at least about any of at 1600 mM alpha-ketoglutaric acid, or the corresponding carboxylate anion, 1700 mM alpha-ketoglutaric acid, or the corresponding carboxylate anion, 1800 mM alpha-ketoglutaric acid, or the corresponding carboxylate anion, 1900 mM alpha-ketoglutaric acid, or the corresponding carboxylate anion, or 2000 mM alpha-ketoglutaric acid, or the corresponding carboxylate anion. In some embodiments, the reaction comprises reacting about 1800 mM alpha-ketoglutaric acid, or the corresponding carboxylate anion. In some embodiments, the reaction is performed starting at a pH of about 6.5 to about 7.5, including about 7. As described herein, the reactions described herein can exhibits changes in pH as the reaction progresses, and thus the description of a starting pH describes when the reactants are brought together for the reacting step. In some embodiments, the reaction comprises reacting cysteine and alpha-ketoglutaric acid, or the corresponding carboxylate anion, at a ratio of about 2:1 to about 1:2, including a ratio of about 1:1. In some embodiments, the reacting is performed for about 5 hours or less, including about any of 4 hours or less, 3 hours or less, 2 hours or less, or 1 hour or less. In some embodiments, the residual concentration of cysteine or cystine in the cysteine source solution is about 0.5 mM or less. In some embodiments, the residual concentration of alpha-ketoglutaric acid, or the corresponding carboxylate anion of any, is about 0.5 mM or less. In some embodiments, the purity of the thiazolidine in the resulting cysteine source solution is at least about 95%, such as at least about any of 96%, 97%, 98%, 99%, or 100%. In some embodiments, the cysteine is L-cysteine.

Provided herein, in certain aspects, is a method of making a cysteine source solution suitable for use with a cell culture, the method comprising: reacting about 1000 mM to about 2500 mM cysteine with about 1000 mM to about 2500 mM oxaloacetic acid, or the corresponding carboxylate anion, in an aqueous solution to produce the cysteine source solution comprising 2-(carboxymethyl) thiazolidine-2,4-dicarboxylic acid, wherein the reaction is performed starting at a pH of about 4 to about 8. In some embodiments, the reaction comprises reacting at least about 1500 mM cysteine, such as at least about any of at 1600 mM cysteine, 1700 mM cysteine, 1800 mM cysteine, 1900 mM cysteine, or 2000 mM cysteine. In some embodiments, the reaction comprises reacting about 1800 mM cysteine. In some embodiments, the reaction comprises reacting at least about 1500 mM oxaloacetic acid, or the corresponding carboxylate anion, such as at least about any of at 1600 mM oxaloacetic acid, or the corresponding carboxylate anion, 1700 mM oxaloacetic acid, or the corresponding carboxylate anion, 1800 mM oxaloacetic acid, or the corresponding carboxylate anion, 1900 mM oxaloacetic acid, or the corresponding carboxylate anion, or 2000 mM oxaloacetic acid, or the corresponding carboxylate anion. In some embodiments, the reaction comprises reacting about 1800 mM oxaloacetic acid, or the corresponding carboxylate anion. In some embodiments, the reaction is performed starting at a pH of about 6.5 to about 7.5, including about 7. As described herein, the reactions described herein can exhibits changes in pH as the reaction progresses, and thus the description of a starting pH describes when the two reactants are brought together for the reacting step. In some embodiments, the reaction comprises reacting cysteine and oxaloacetic acid, or the corresponding carboxylate anion, at a ratio of about 2:1 to about 1:2, including a ratio of about 1:1. In some embodiments, the reacting is performed for about 5 hours or less, including about any of 4 hours or less, 3 hours or less, 2 hours or less, or 1 hour or less. In some embodiments, the residual concentration of cysteine or cystine in the cysteine source solution is about 0.5 mM or less. In some embodiments, the residual concentration of oxaloacetic acid, or the corresponding carboxylate anion of any, is about 0.5 mM or less. In some embodiments, the purity of the thiazolidine in the resulting cysteine source solution is at least about 95%, such as at least about any of 96%, 97%, 98%, 99%, or 100%. In some embodiments, the cysteine is L-cysteine.

In some embodiments, provided is a method comprising preparing a cysteine source solution according to the methods provided herein, preparing (in any sequences or in parallel) a cell culture medium (such as preparing a cell culture medium in a cell culture tank), adding an amount of the cysteine source solution to the cell culture medium, QSing the cell culture medium with the cysteine source solution, and performing sterile filtration on the cell culture medium with the cysteine source solution.

### C. Additional Compositions Comprising a Cysteine Source Solution and Uses of the Same

The inventions provided herein also encompass processes including the reactions for producing cysteine source solutions (as well as the cysteine source solutions and derivatives produced therefrom) and aspects enabled by the use of the taught cysteine source solutions.

In some embodiments, provided is a method of method of making a cell culture medium, the method comprising admixing a basal cell culture medium with a cysteine source solution (including a dried form thereof) to make the cell culture medium, wherein the cysteine source solution is produced according to any method described herein. In some embodiments, the cell culture medium is suitable for cysteine-dependent cells. In some embodiments, the basal cell culture medium is not suitable for cysteine-dependent cells without further modification, e.g., without addition of a sufficient amount of cysteine (or alternatively, a cysteine source solution taught herein). In some embodiments, the basal cell culture medium has a pH of about 5 to about 8. In some embodiments, the method further comprises preparing the thiazolidine side solution according to a method described herein. In some embodiments, the method further comprises adjusting the pH of the basal cell culture medium. In some embodiments, the method further comprises QSing the cell culture medium after admixing the basal cell culture medium and the cysteine side solution. In some embodiments, the basal cell culture medium is substantially free of cysteine and cystine (such as not containing an amount of a cysteine and/or cystine to enable growth and maintenance of a cysteine-dependent cell).

In some embodiments, provided herein is a method of culturing a cysteine-dependent cell, the method comprising culturing the cysteine-dependent cell in a cell culture medium made according to any method provided herein. In some embodiments, the method of culturing comprises maintaining a level of a thiazolidine by further subjecting the cell to a cysteine source solution (such as by directly adding the cysteine source solution to a cell culture medium comprising the cell and/or providing the cell with fresh cell culture medium comprising the cysteine source solution).

In some embodiments, provided herein is a method of producing a polypeptide, the method comprising culturing a cysteine-dependent cell in a cell culture medium made according to any method described herein, and obtaining the polypeptide therefrom. In some embodiments, the polypeptide is a therapeutic polypeptide or a precursor thereof. In some embodiments, the therapeutic polypeptide or the precursor thereof is an antibody or a fragment thereof. In some embodiments, the antibody or a fragment thereof is an antibody drug conjugate, or a precursor thereof that is conjugated following production. It is noted that the inventors have further contemplated the advantage of using the present methods for making drug conjugates, including the lack of or low levels of cysteine available to binding to cysteine residues of the polypeptide, thus greatly facilitating the production of drug conjugates as compared to using manufacturing techniques involving cysteine containing cell culture media.

The inventions provided herein are relevant to many cell types, and particularly culturing cysteine-dependent cells. Cell culture media are well-known in the art and may include or require addition of a cysteine source. The instant application provides a means for preparing a suitable cell culture medium using a thiazolidine and does not require the use of cysteine or a form thereof, such as cystine. In some embodiments, the cell culture medium is produced using a basal cell culture medium deficient of a sufficient amount of a cysteine source, and adding a cysteine source solution taught herein. Media, and components needed to support cell growth, are described in Ham et al., Meth Enz, 58, 1979, Barnes et al., Anal Biochem, 102, 1980, U.S. Pat. Nos. 4,767,704; 4,657,866; 4,927,762; 4,560,655; or 5,122,469; International Patent Application Nos. WO 90/03430 or WO 87/00195; and U.S. Patent Reissue No. 30,985, which are hereby incorporated herein by reference in their entirety. Any of these media may be further supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. In some embodiments, the culture medium further comprises a glucose source. In some embodiments, the culture medium further comprises a mannose source. Any other necessary supplements may also be included at appropriate concentrations that would be known to those skilled in the art.

Methods of culturing a cell in a culture medium are well known to those in the art. See, e.g., Li et al., MAbs, 2, 2010. In some embodiments, the culturing technique is a fed-batch culture process. In some embodiments, the culturing technique is a batch culture process. In some embodiments, the culturing technique is a continuous culture process. In some embodiments, the culturing technique is a perfusion culture process.

In some embodiments, the culturing comprises performing a reaction described herein to produce a cysteine source solution, admixing the cysteine source solution and a basal cell culture medium lacking a sufficient cysteine source to support growth and maintenance of a cysteine-dependent cell, sterile filtering the produced cell culture medium, and providing the cell culture medium to a cell as needed during the culturing process. In some embodiments, the culturing technique is a fed-batch culture process. In some embodiments, the culturing technique is a batch culture process. In some embodiments, the culturing technique is a continuous culture process. In some embodiments, the culturing technique is a perfusion culture process. In some embodiments, the method further comprises obtaining a product produced by the cell, e.g., a polypeptide.

The culture conditions, such as temperature, pH, and the like, will be apparent to the ordinarily skilled artisan. For example, generally, the production of proteins is done on a large scale (such as a commercial scale). To achieve a population of a cell suitable for commercial scale production, one of ordinary skill in the art will recognize the utility using a stepwise approach to expanding a cell population. For example, the process involves growing a desired cell on a smaller scale to allow for an increase in the cell population, such as a seed train. To further increase the population of the cell, methods generally involved using the seed train to inoculate a larger culture tank, such as an inoculum tank or a bioreactor. This process will provide a suitable population of a cell for culture in a production culture. In some embodiments, the production culture is a 1000 L culture tank. The cysteine source solutions provided herein may be used for any one or more of the culturing steps. In some embodiments, the cell culture is maintained at a specified temperature. In some embodiments, the specified temperature is about 15° C. to about 45° C. In some embodiments, the specified temperature is about 30° C. In some embodiments, the specified temperature is less than about 37° C. In some embodiments, the specified temperature is less than about 35° C. In some embodiments, the specified temperature is less than about 34° C. In some embodiments, the cell culture is maintained at a specified pH. In some embodiments, the cell culture is maintained at a specified dissolved oxygen concentration. In some embodiments, the cell culture is maintained at a specified nutrient level.

In some embodiments, provided herein is a method of culturing a cell, the method comprising providing a cell culture medium to the cell, wherein the cell culture medium comprises a basal culture medium and a cysteine source solution comprising a thiazolidine, and wherein and the cell culture medium does not comprise more than about 20% cysteine relative to the amount of the thiazolidine in the cell culture medium; and culturing the cell. In some embodiments, the cell is a cysteine-dependent cell. In some embodiments, the cell culture medium is not suitable for culturing the cell without further including cysteine or a source thereof. In some embodiments, the cysteine source solution is the sole source of cysteine for the cell. In some embodiments, the cell culture medium does not comprise cysteine or cystine. In some embodiments, the method of culturing the cell is a fed-batch technique performed in a bioreactor. In some embodiments, the cell culture comprises a fed-batch cell culture in a bioreactor. In some embodiments, the fed-batch cell culture is carried out for a duration of 1 to 21 days, such as any of 1 days, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In some embodiments, the cysteine source solution has a concentration of the thiazolidine of about 5 mM to about 50 mM, such as about any of 5 mM, 10 mM, 15 mM, 20 mM, 25 mM, 30 mM, 35 mM, 40 mM, 45 mM, or 50 mM. In some embodiments, the cysteine source solution is added to the bioreactor daily. In some embodiments, the cysteine source solution is added throughout the fed-batch cell culture process. In some embodiments, the fed-batch cell culture process is a viable cell density-dependent strategy. In some embodiments, the volume of cysteine source solution added to the bioreactor is based on the viable cell density. In some embodiments, the fed-batch cell culture is seeded at a cell density of about 1×10⁶ to about 8×10⁶ viable cells/mL. In some embodiments, the peak density of viable cells is 15×10⁶ to about 50×10⁶ cells/mL. In some embodiments, the viable cell density is about 15×10⁶ to about 50×10⁶ viable cells/mL. In some embodiments, the daily feeding volumes are about 0.5% to about 8.5% of the working volume per day. Techniques for measuring viable cell density are known in the art and include techniques such as the trypan blue exclusion assay and permittivity measurements, e.g., Riss et al., Cell Viability Assays, The Assay Guidance Manual, 2016, and Rosner et al., Bioengineering, 9, 2022, which are hereby incorporated herein by reference in their entirety. In some embodiments, the method further comprises harvesting when the cell viability in the cell culture drops to about 70% to about 80%. In some embodiments, the daily feeding volumes are about 0.5% to about 8.5% of the working volume per day. In some embodiments, the cell is a mammalian host cell that is transformed with a gene encoding a biomolecule of interest. In some embodiments, the mammalian host cell is a CHO cell, a HeLa cell, a HEK293 cell, a Vero cell. In some embodiments, the mammalian host cell is a CHO cell. In some embodiments, the biomolecule comprises an antibody or an antigen-binding fragment thereof, an enzyme, a viral vector comprising a therapeutic transgene, or a recombinant enzyme. In some embodiments, the antibody or an antigen-binding fragment thereof comprises a monoclonal antibody (mAb), a bispecific antibody, a trispecific antibody, or an immunoglobulin single variable domain.

Cells relevant to the description provided herein generally require a cysteine source for culturing. Conventionally, such cysteine source is provided by, e.g., cysteine in the cell culture medium. Generally speaking, relevant cells include eukaryotic cells, such as yeast or higher eukaryotic cells, and such higher eukaryotic cells include insect cells and established cell lines of mammalian origin. Examples of suitable mammalian cells include the COS-7 line of monkey kidney cells (ATCC CRL 1651) (Gluzman et al., Cell, 23, 1981), L cells, 293 cells, C127 cells, 3T3 cells (ATCC CCL 163), Chinese hamster ovary (CHO) cells, or their derivatives such as Veggie CHO and related cell lines which grow in serum-free media (Rasmussen et al., Cytotechnology, 28, 1998), HeLa cells, BHK (ATCC CRL10) cell lines, and the CVI/EBNA cell line derived from the African green monkey kidney cell line CVI (ATCC CCL 70) as described by McMahan et al., EMBO J, 10, 1991, human embryonic kidney cells such as 293, 293 EBNA, or MSR 293, human epidermal A431 cells, human Colo205 cells, other transformed primate cell lines, normal diploid cells, cell strains derived from in vitro culture of primary tissue, primary explants, HL-60, U937, HaK, or Jurkat cells. Optionally, for example, mammalian cell lines such as HepG2/3B, KB, NIH 3T3 or S49, can be used as cells for the methods provided herein.

In some embodiments, the cell is a CHO cell. CHO cells are well known in the art. See, e.g., Xu et al., Nat Biotechnol, 29, 2011. In some embodiments, the cell is a DP12 host cell. In some embodiments, the hst cell is a DUXB-11 derived DHFR-deficient DP12 cell. In some embodiments, the cell is a CHO-K1 host cell. In some embodiments, the cell is a DHFR-positive CHO-K1 host cell. In some embodiments, the cell is a CHOK1M cell.

In some embodiments, the cell is a mouse host cell. In some embodiments, the cell is a Sp2/0 host cell. In some embodiments, the cell is a NSO host cell.

In some embodiments, the cell is a hybridoma. In some embodiments, the hybridoma is an antibody-producing cell, wherein the antibody-producing cell is collected from a host following immunization of the host with an antigen. In some embodiments, the antibody-producing cell is fused with a myeloma cell. In some embodiments, the cell is a mouse myeloma-derived cell line.

Alternatively, the cell can be a lower eukaryote such as yeast. Suitable yeasts include Saccharomyces cerevisiae, Schizosaccharomyces pombe, Kluyveromyces strains, Candida, or any yeast strain capable of expressing heterologous polypeptides.

As described herein, the methods provided may be used to produce a product from a cell, such as a polypeptide. In some embodiments, provided herein is a method of producing a polypeptide, the method comprising culturing a cysteine-dependent cell in a cell culture medium made according to any embodiment provided herein, and obtaining the polypeptide therefrom. In some embodiments, the polypeptide is a therapeutic polypeptide or a precursor thereof. In some embodiments, the therapeutic polypeptide or the precursor thereof is an antibody or a fragment thereof. In some embodiments, the antibody or a fragment thereof is an antibody drug conjugate.

In some aspects, provided herein are methods using a cell culture comprising a cysteine source solution comprising a thiazolidine described herein, including a concentrated cell culture medium. Exemplary methods using feeds taught herein include fed-batch culturing techniques, perfusion culturing techniques, and in-line dilution culturing techniques. The culture media described herein may be used on a variety of scales including small research scale cell cultures to commercial scale production cultures.

### D. Kits, Components, Compositions, and Systems

Also provided herein, in other aspects, are kits, components, compositions, and systems comprising the cysteine source solutions (or components of the described reactions). For example, in some embodiments, provided herein are kits comprising components to perform the reactions taught herein. In some embodiments, provided herein is a kit comprising a cysteine source solution, or a precursor thereof, produced according to the methods described herein. In some embodiments, the kit comprises instructions for use according to the methods described herein. In some embodiments, provided herein is a cysteine source solution produced according to the methods described herein. In some embodiments, provided is a cell culture medium comprising a cysteine source solution produced according to the methods described herein. In some embodiments, the cysteine source solution and/or cell culture medium is sterile (such as accomplished by sterile filtration of the same).

In certain aspects, provided herein is a concentrated basal cell culture medium comprising a thiazolidine described herein, e.g., as synthesized according to the methods described herein. In some embodiments, the concentrated basal cell culture medium is about 2× to about 10×, such as about any of 2×, 3×, 4×, 5×, 6×, 7×, 8×, 9×, or 10×. In some embodiments, the basal cell culture medium is substantially free of cysteine or cystine, such as comprises 2% or less of cysteine or cystine.

In some embodiments, provided herein is a cell culture system comprising: a cell; and a basal cell culture medium admixed with the cysteine source solution provided herein. In some embodiments, the basal cell culture medium is substantially free of cysteine and cystine.

### EXAMPLES

### Example 1: Materials and Methods

### Thiazolidine Synthesis

Cysteine (Cys) (CAS: 52-90-4, Sigma-Aldrich, Cat #C₇₃₅₂) and ketoacids were mixed in aqueous solution with different pH, concentrations and ratios at ambient condition (room temperature) to form thiazolidines (cysteine source solutions). Three ketoacids were used in the synthesis method optimization: sodium pyruvate (Pyr) (CAS: 113-24-6, ThermoFisher Scientific, Cat #AAJ6184018), α-Ketoglutaric Acid (AKG) (CAS: 328-50-7, Sigma-Aldrich, Cat #K1128), and Oxaloacetic Acid (Oxa) (CAS: 328-42-7, Sigma-Aldrich, Cat #07753).

### DTNB Solution:

The DTNB assay was employed for the indirect measurement of thiazolidine synthesis. The conversion of cysteine (Cys) into thiazolidine occurs through the reaction with a ketoacid, resulting in the formation of a sulfur-ring structure. The disappearance of free thiol groups in the reaction was quantified using the DTNB assay, with the readout achieved through absorbance measurements at 412 nm.

DTNB solution was prepared by dissolving 2.5 mM 5,5′-dithio-bis-(2-nitrobenzoic acid) (CAS: 69-78-3, ThermoFisher Scientific, Cat #22582) in 0.5 M Tris buffer. Strong buffer was used as DTNB will decompose outside of the pH range of 7-8. The solution was used the day it was made and was stored in the fridge when not in use.

When preparing the dilution, the primary objective was to achieve the optimal thiol concentration for the DTNB assay and to ensure that the pH of the experimental solution fell within the ideal range of 7-8. Thiol solution samples were diluted with Tris-buffer to a final concentration of 1 mM maximum and the pH of the solution was measured and kept at the proper range for the assay.

50 μL of the diluted samples was mixed with 150 μL of the DTNB solution within the wells of a 96-well plate with a clear bottom. The contents were mixed by carefully pipetting up and down, ensuring that no bubbles formed in the wells. A control well was prepared with 50 μL of deionized water (RODI) and 150 μL of the DTNB solution to serve as the blank reference. A fresh blank for each time point was prepared. This practice ensured that any potential decomposition in the DTNB stock was properly accounted for during absorbance measurements and maintained the accuracy of the assay. The resulting well plate was measured with SpectraMax plate reader at a absorbance at 412 nm.

### LC-MS:

LC-MS analysis was employed for the direct measurement of thiazolidine synthesis to confirm the purity of the thiazolidines. LC-MS analysis was performed using a Thermo Q-Exactive Plus Orbitrap mass spectrometer (Thermo Fisher Scientific, San Jose, USA) equipped with a heated electrospray ionization source (HESI-II) and a Vanquish UPLC system. The LC separation was achieved by injecting 10 microliters of either sample or standard solution into a Hypercarb porous graphitic carbon HPLC column with 4.6 mm i.d.×100 mm dimension and 4.6 μm particle size (Thermo Fisher Scientific, San Jose, USA. Part No. 35003-104630). The mobile phase A was 20 mM NH4Formate, 0.1% FA, Water, and mobile phase B was 20 mM NH4Formate, 0.1% FA, 90% MeOH: water. The method was set at a gradient from 10% to 80% B from 0 to 10 min, followed by a gradient from 80% to 100% B from 10 min to 12 min and held isocratic until 18 min, then held at isocratic of 0% B from 18 min to 20 min. The total run time was 20 min with a flow rate of 0.5 mL/min. Samples were analyzed in full scan MS mode from 60 to 900 m/z under positive polarity with electrospray ionization. The resolution of 70,000 FWHM with 3.0×10⁶ of Automatic Gain Control (AGC) target and 100 ms of maximum ion injection time was fixed during the analysis. Software used for operating the LC-HRMS was Xcalibur™ (version 4.1).

For data-dependent acquisition (DDA), the mass spectrometer was operated in positive ion mode, the survey scans were acquired at a resolution of 70,000 with a maximum injection time of 100 ms and target value of 3.0×10⁶ over mass range of 60 to 900 m/z. The target candidate masses of 192.0322, 250.0376 and 236.0220 Daltons were added into inclusion lists. The fragments were recorded at a resolution of 35,000 with an injection time of 50 ms and a target value of 1×10⁵. The isolation window was set at 4 m/z and the normalized collision energy (NCE) was at 35.

### Fed-Batch Cell Culture:

In-house cell culture media and feed were used in the cell culture experiments. The process was performed in an Ambr 15 with up to 48 disposable cell culture bioreactor vessels (Sartorius Stedim Biotech GmbH), or 3 L stir-tank bioreactor with DeltaV controller. Ambr15 cultivations were performed using a platform strategy: agitation at 1400 rpm, DO at 40% of air saturation, pH controlled ≥6.9, and temperature shift from 36.5° C. to 34° C. on Day 4. To prevent foaming, 20 μL of FoamAway™ Irradiated AOF (animal origin-free) Antifoaming Agent was added every 48 h. The 3 L bioreactor cultivations were performed using an intensified process: inoculation VCD target at 10 Mvc/mL, agitation at 250 rpm, DO at 40% of air saturation, pH controlled ≥6.9, and temperature shift from 36.5° C. to 34° C. on Day 2.

### Example 2: Optimization of Thiazolidine Production

### Optimization Test 1

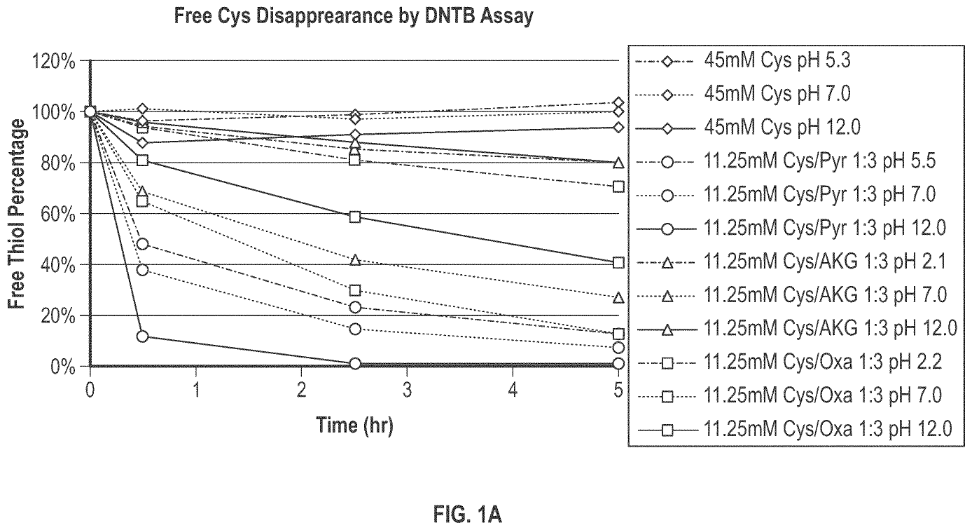

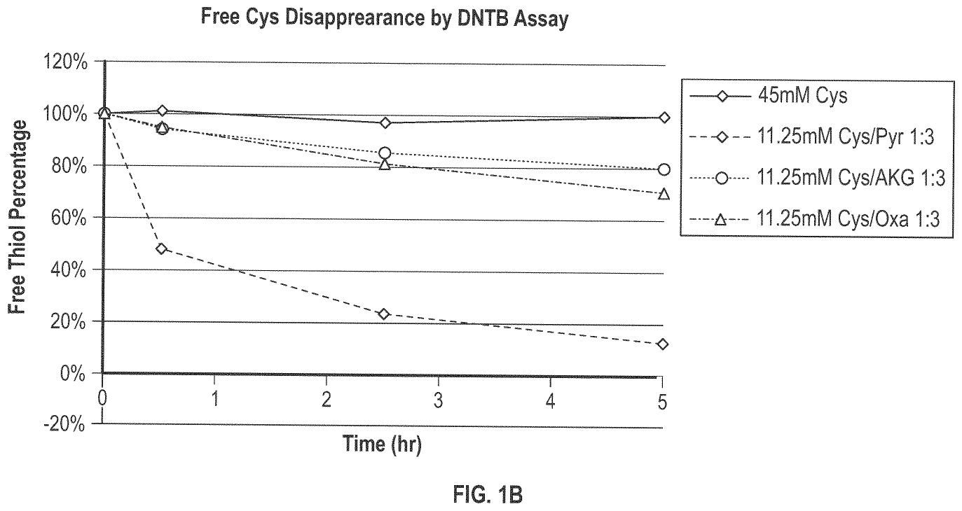

Fresh Cys solution was mixed with Fresh Pyr (Cys/Pyr), AKG (Cys/AKG), and Oxa (Cys/Oxa) solution, respectively, at 1:3 ratio with Cys at a final concentration of 11.25 mM. The three mixture conditions were at acidic pH with initial pH at 5.5, 2.1, and 2.2, respectively. In addition, the three mixture conditions were adjusted the initial pH to 7.0 and 12.0, respectively, by NaOH solution titration. DTNB assay was conducted for each mixture condition at T=0.5, 2.5 and 5 h to monitor free Cys disappearance rates, which corresponds to the thiazolidine synthesis rates. See FIG. 1A and Table 1. The results revealed that Cys/Pyr mixtures had the highest synthesis rate, while Cys/AKG and Cys/Oxa mixtures followed in sequence. Cys/Pyr demonstrated its highest synthesis rate at pH 12, whereas Cys/AKG and Cys/Oxa exhibited their highest rates at pH 7.

TABLE 1
Cys disappearance by DTNB assay in Test 1

| | 45 mM Cys | | | 11.25 mM Cys/Pyr 1:3 | | | 11.25 mM Cys/AKG 1:3 | | | 11.25 mM Cys/Oxa 1:3 | | |
|---|---|---|---|---|---|---|---|---|---|---|---|---|
| Time | pH | pH | pH | pH | pH | pH | pH | pH | pH | pH | pH | pH |
| (hr) | 5.2 | 7.0 | 12.0 | 5.5 | 7.0 | 12.0 | 2.1 | 7.0 | 12.0 | 2.2 | 7.0 | 12.0 |
| 0 | 100% | 100% | 100% | 100% | 100% | 100% | 100% | 100% | 100% | 100% | 100% | 100% |
| 0.5 | 101% | 97% | 88% | 48% | 38% | 12% | 94% | 69% | 96% | 95% | 65% | 81% |
| 2.5 | 97% | 98% | 92% | 23% | 15% | 1% | 86% | 42% | 88% | 82% | 30% | 59% |
| 5 | 100% | 103% | 94% | 12% | 7% | 1% | 80% | 27% | 80% | 71% | 13% | 41% |

### Optimization Test 2

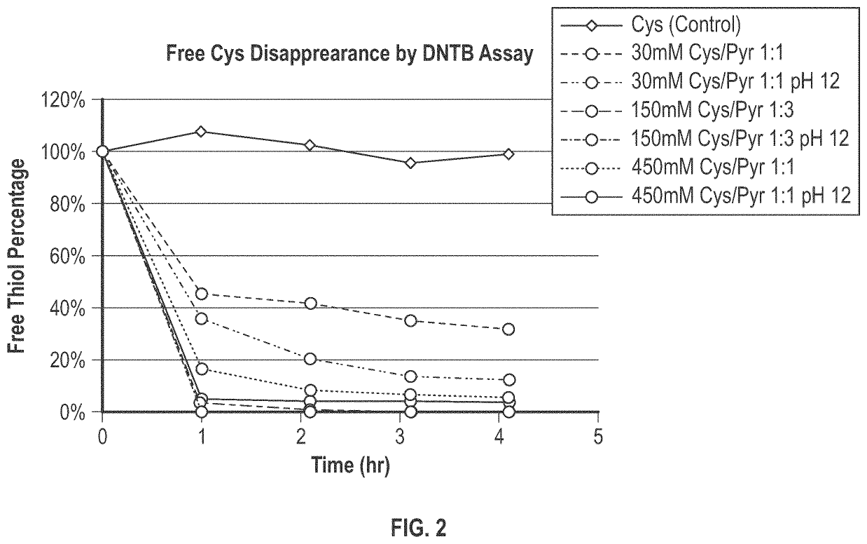

The substrate concentration and ratio for the thiazolidine synthesis were optimized using Cys/Pyr as an example. The test involved three concentration conditions: 30 mM of Cys mixed with Pyr at a 1:1 ratio, 150 mM of Cys mixed with Pyr at a 1:3 ratio, and 450 mM of Cys mixed with Pyr at a 1:1 ratio. All three conditions were tested at initial pH of 4.6 and 12. A DTNB assay was conducted for each mixture condition at T=1, 2, 3 and 4 h to monitor free Cys disappearance rates, which correspond to the thiazolidine synthesis rates. See FIG. 2 and Table 2. The results showed that higher substrate concentrations of Cys/Pyr had higher synthesis rate and that higher substrate concentrations can help balance the substrate ratio of Cys/Pyr to 1:1 with decent synthesis rates. For the Cys/Pyr mixture, pH adjustment to an initial pH of 12 demonstrated a higher synthesis rate than original initial pH of 4.6, which aligns with the trend of pH effect discovered in Test 1.

TABLE 2
Cys disappearance by DTNB assay in Test 2

| Time | 30 mM | 30 mM Cys/Pyr 1:1 | | 150 mM Cys/Pyr 1:3 | | 450 mM Cys/Pyr 1:1 | |
|---|---|---|---|---|---|---|---|
| (hr) | Cys | pH 4.6 | pH 12 | pH 4.6 | pH 12 | pH 4.6 | pH 12 |
| 0 | 100% | 100% | 100% | 100% | 100% | 100% | 100% |
| 1 | 108% | 45% | 36% | 3% | 0% | 17% | 5% |
| 2.1 | 103% | 42% | 20% | 0% | 0% | 8% | 4% |
| 3.1 | 96% | 35% | 14% | 1% | 1% | 7% | 4% |
| 4.1 | 99% | 32% | 12% | 0% | 1% | 5% | 4% |

### Optimization Test 3

In order to develop an optimal synthesis process for all three thiazolidines of Cys/Pyr, Cys/AKG and Cys/Oxa, an initial pH of 7 (achieved by titrating to neutral pH titrated by NaOH) with high initial concentration was chosen for further optimization testing. Based on the trends related to pH that were observed in Optimization Test 1, an initial pH of 7 should yield the highest synthesis rate as compared to acidic or basic pH for Cys/AKG and Cys/Oxa.

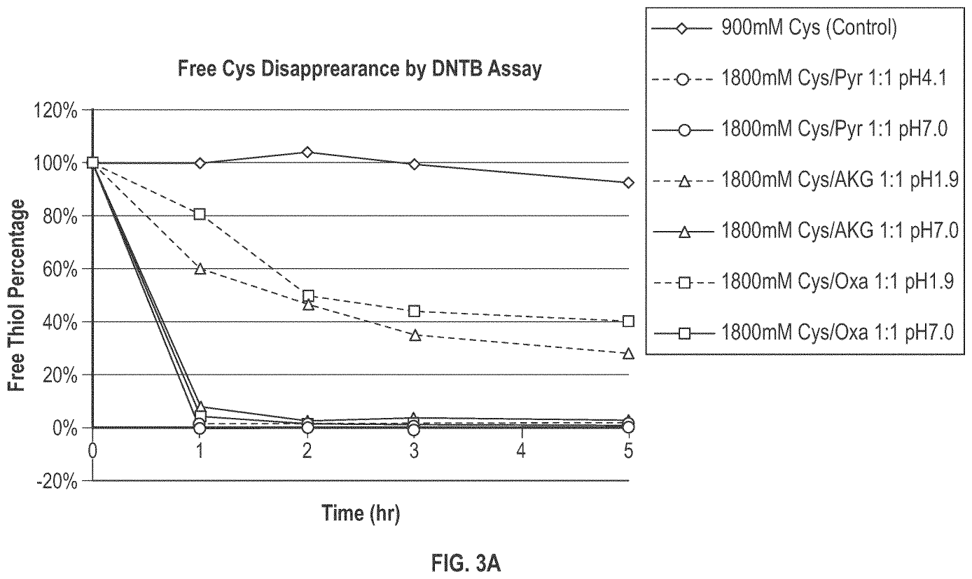

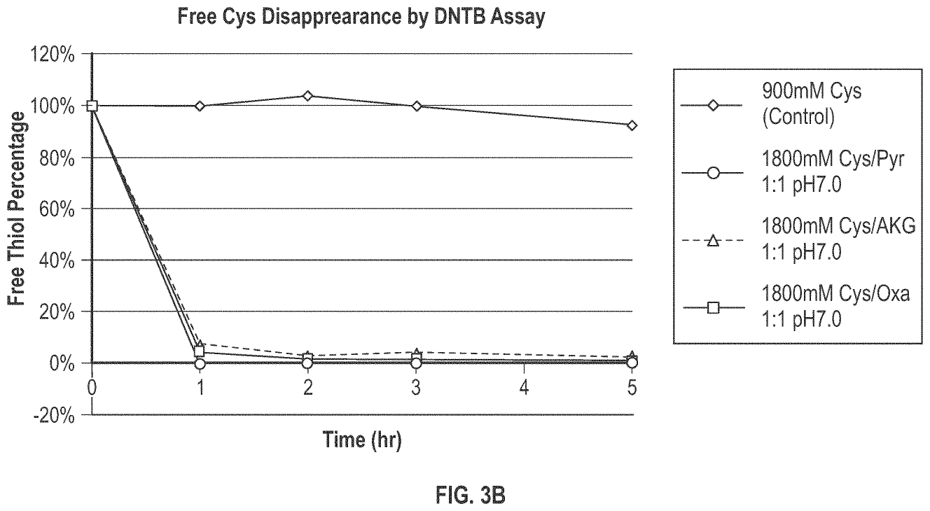

The initial concentration of Cys was increased to 1800 mM, and mixed with Pyr (Cys/Pyr), AKG (Cys/AKG), or Oxa (Cys/Oxa) at concentration ratio of 1:1 with either acidic or neutral pH adjusted by NaOH. A DTNB assay was conducted for each mixture condition at T=1, 2, 3 and 5 h to monitor free Cys disappearance rates, which correspond to the thiazolidine synthesis rates. See FIG. 3A and Table 3. The results showed all three reactions of substrates at 1:1 ratio for thiazolidines of Cys/Pyr, Cys/AKG, and Cys/Oxa almost completed in one hour with high initial concentration of 1800 mM at initial pH of 7.

TABLE 3
Cys disappearance by DTNB assay in Test 3

| Time | 900 mM | 1800 mM Cys/Pyr 1:1 | | 1800 mM Cys/AKG 1:1 | | 1800 mM Cys/Oxa 1:1 | |
|---|---|---|---|---|---|---|---|
| (hr) | Cys | pH 4.1 | pH 7.0 | pH 1.9 | pH 7.0 | pH 1.9 | pH 7.0 |
| 0 | 100% | 100% | 100% | 100% | 100% | 100% | 100% |
| 1 | 100% | 1% | 0% | 60% | 8% | 81% | 4% |
| 2 | 104% | 1% | 0% | 47% | 3% | 51% | 1% |
| 3 | 100% | 0% | 0% | 35% | 4% | 44% | 1% |
| 5 | 93% | 0% | 0% | 28% | 3% | 40% | 1% |

The pH effects and the concentration effects on thiazolidine synthesis rates were consistent between Optimization Tests 1, 2, and 3. Thus, it was determined that 1:1 ratios of Cys: α-ketoacid, a concentration range of 900-1800 mM, and a pH of 7 are the optimal initial conditions for aqueous conversion of Cys to thiazolidine with high purity.

As the differences shown between FIG. 1B and FIG. 3B, pH, concentration, and ratio optimization led to a significant reduction in the reaction time, e.g., complete reaction was obtained in about 1 hour following optimization (FIG. 3B), and it was observed that the conditions in FIG. 3B with a 1:1 ratio of substrates resulted in a pure product (cf with FIG. 1B where it was observed that excess alpha-ketoacid was present following the reaction).

### Example 3: Confirmation of Synthesized Thiazolidine with High Purity and Stability

### High-Purity Thiazolidine Solutions

Thiazolidine solutions (cysteine source solutions) were prepared using the optimal process and added into the mammalian cell culture feeds during media formulation. LC-MS and HPLC methods were used to confirm the thiazolidine structures in the solutions and their associated purity.

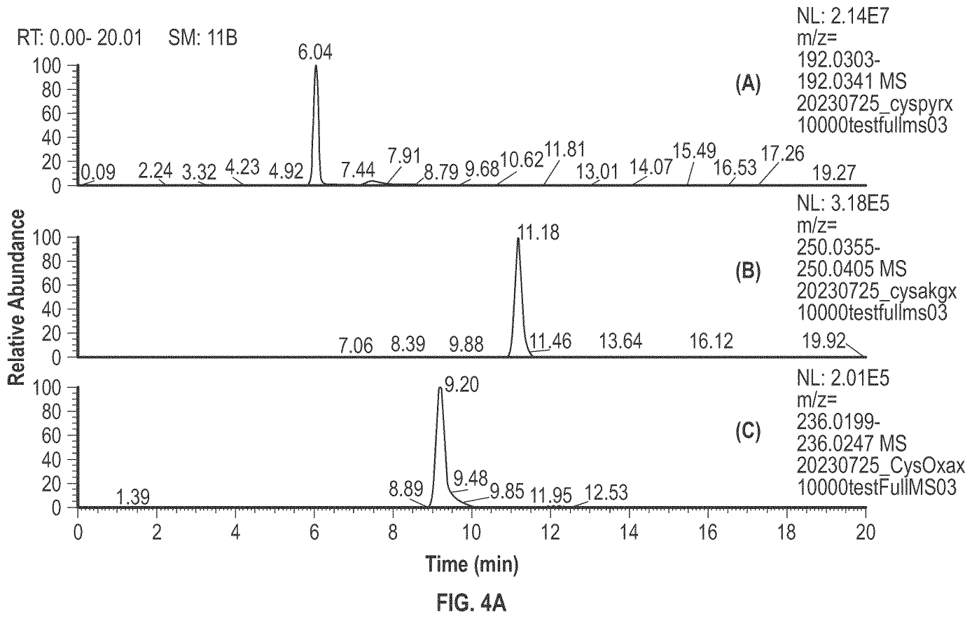

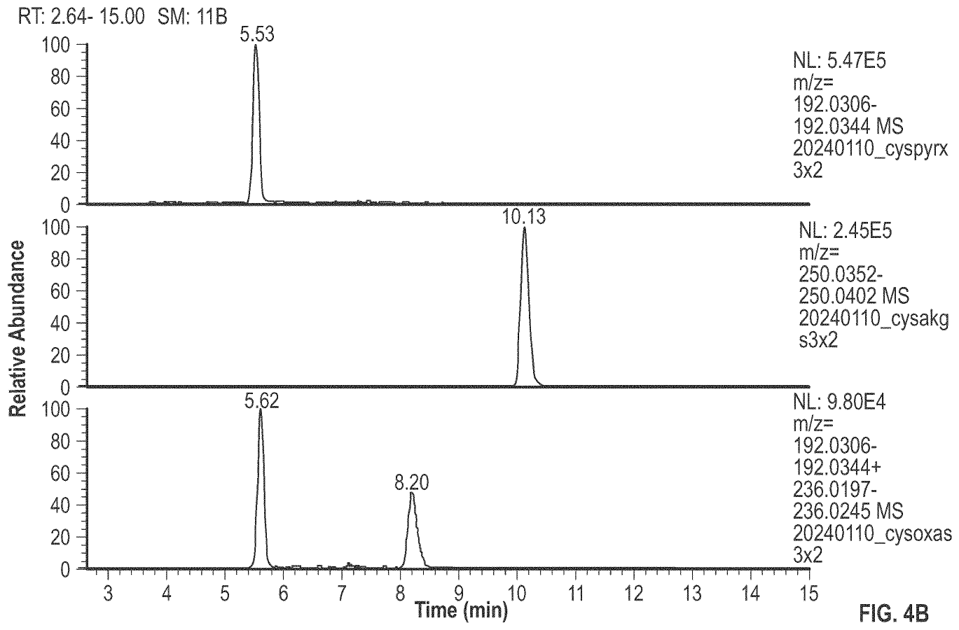

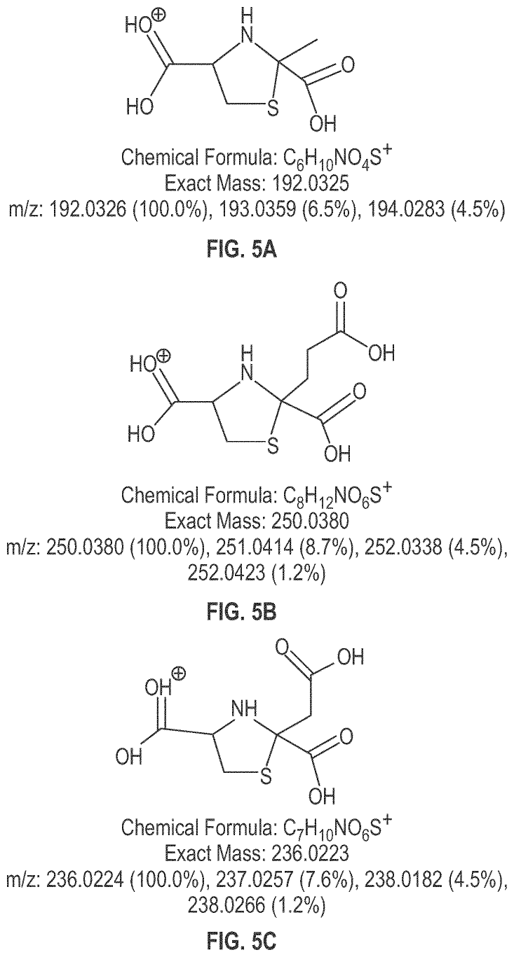

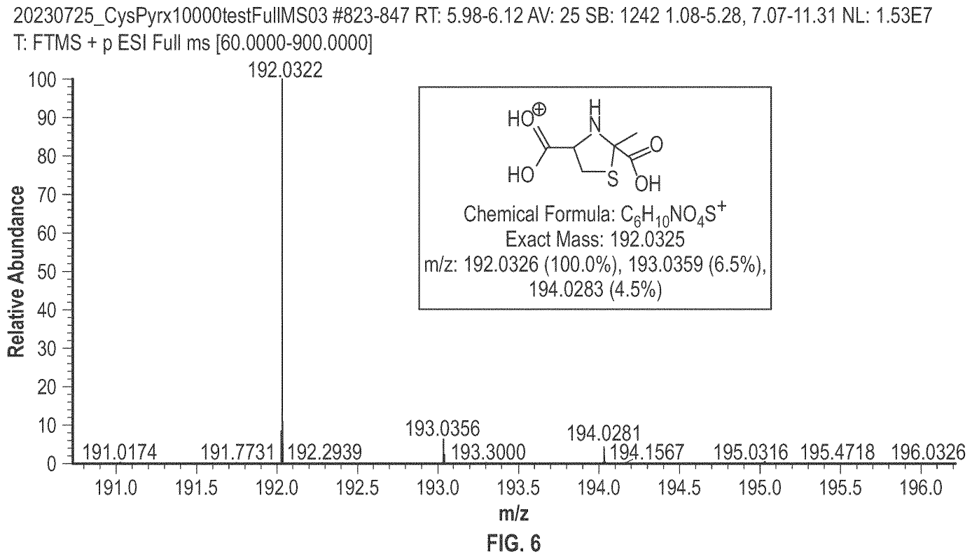

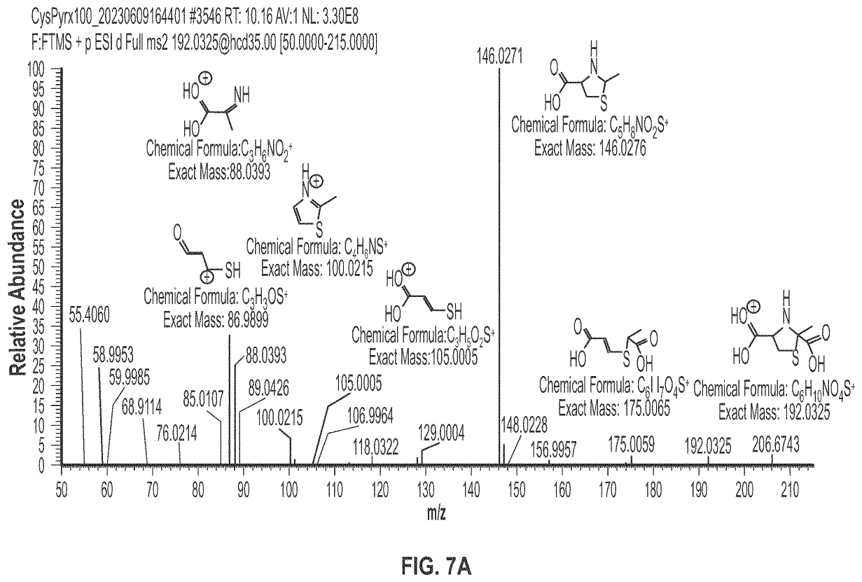

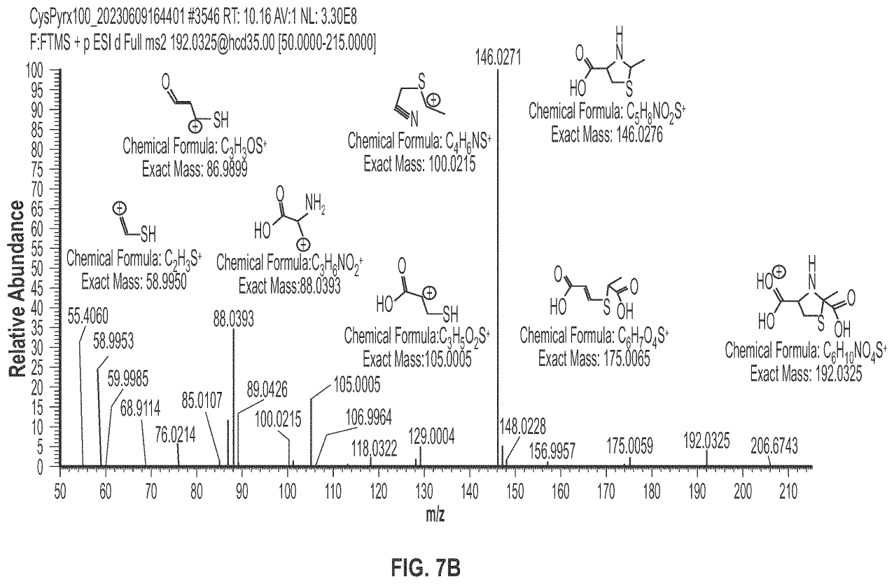

The sample solutions were diluted 100 times with mobile phase A, and LC peaks eluted at retention time of 6.04, 11.18 and 9.20 min for thiazolidine samples of Cys-Pyr, Cys-AKG and Cys-Oxa, respectively (FIG. 4A). A replicate analysis was performed and the chromatograms for thiazolidine samples of Cys-Pyr, Cys-AKG and Cys-Oxa are provided in FIG. 4B. Associated mass spectra were obtained for Cys-Pyr, Cys-AKG, and Cys-Oxa (data not provided). In the replicate analysis, it was observed that some amount of Cys-Oxa is converted to Cys-Pyr. The chemical structures of proposed complexes are shown in FIG. 5, the theoretical exact mass of each molecule was calculated using Software ChemDraw Professional (Version 22.2.0.3300). Protonated molecular weights at 192.0322, 250.0376, 236.0220 Daltons were identified to be the three complexes of interest. For Cys-Pyr, the most intensive peak of 192.0322 m/z in the high-resolution mass spectra (FIG. 6) matched with the theoretical monoisotopic mass of 192.0325 for C₆H₁₀NO₄S⁺, while the remaining peaks also appeared to be isotopic matches. However, the detailed structures could not be assigned by this result since it does not provide the arrangement of atoms within the molecule. To further elucidate the structure, tandem mass spectrometry was used to provide fragmentation patterns to help the interpretation. Thus, 192.0325 m/z with the retention time at 6.04 min was selected as the parent ion for DDA. Structures of daughter ions were predicted using ChemDraw. The spectrum result is shown in FIGS. 7A and 7B, with predicted fragment structures applied to the tandem mass spectrum shown therein.

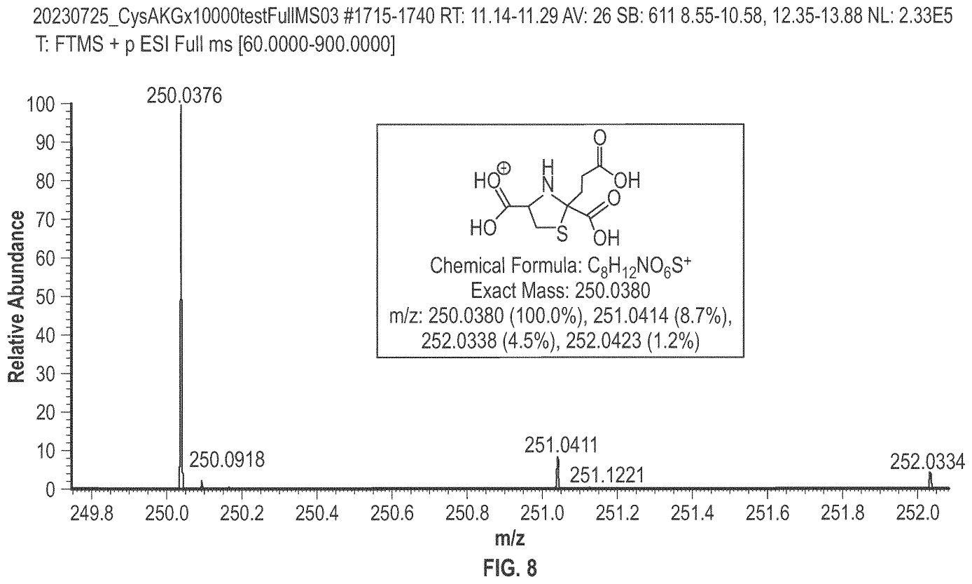

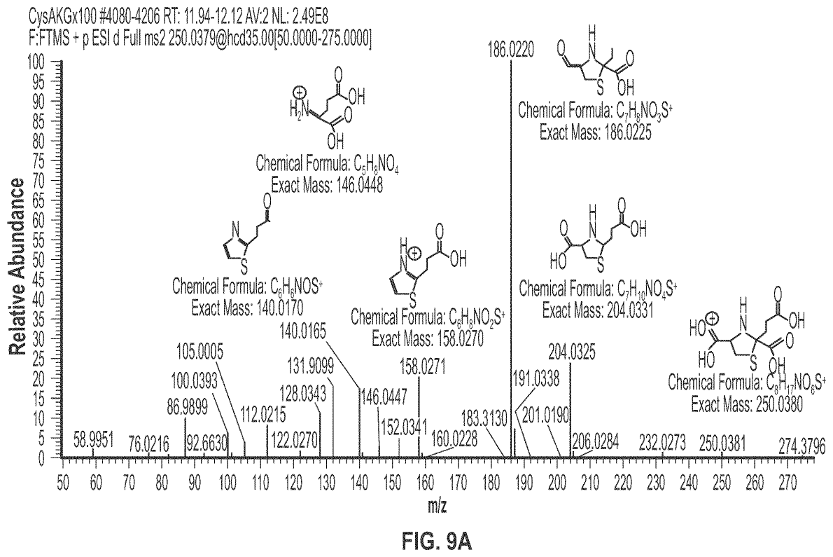

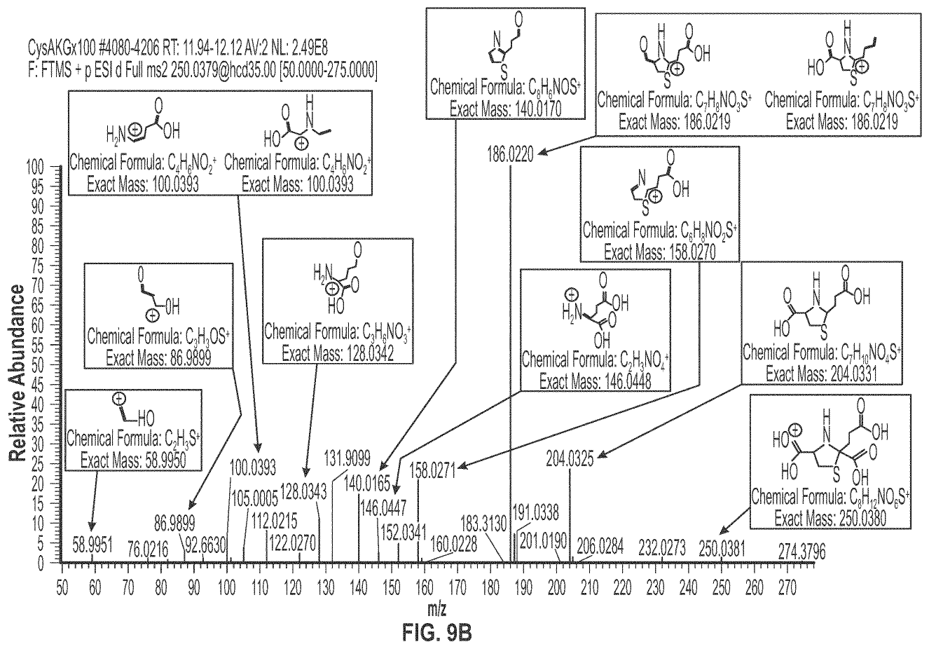

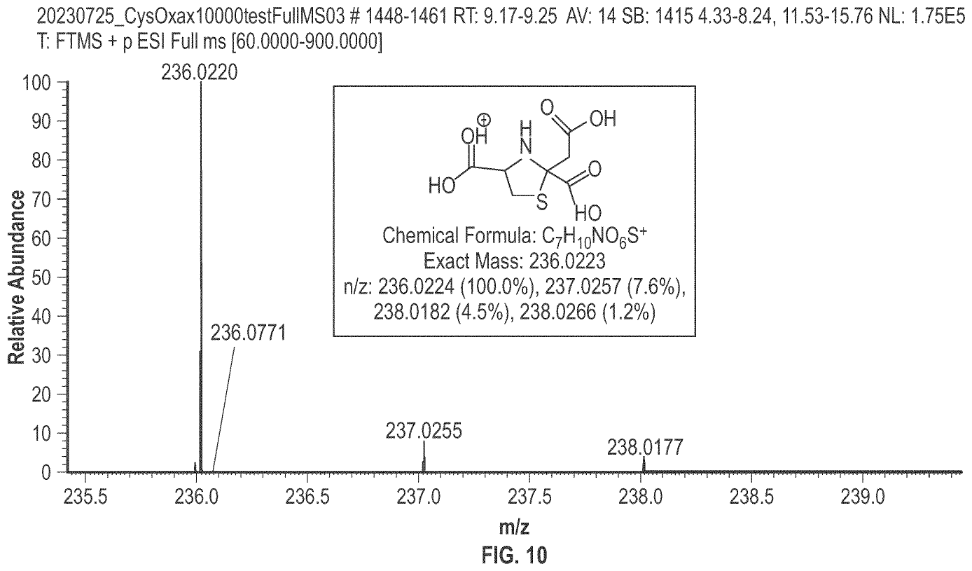

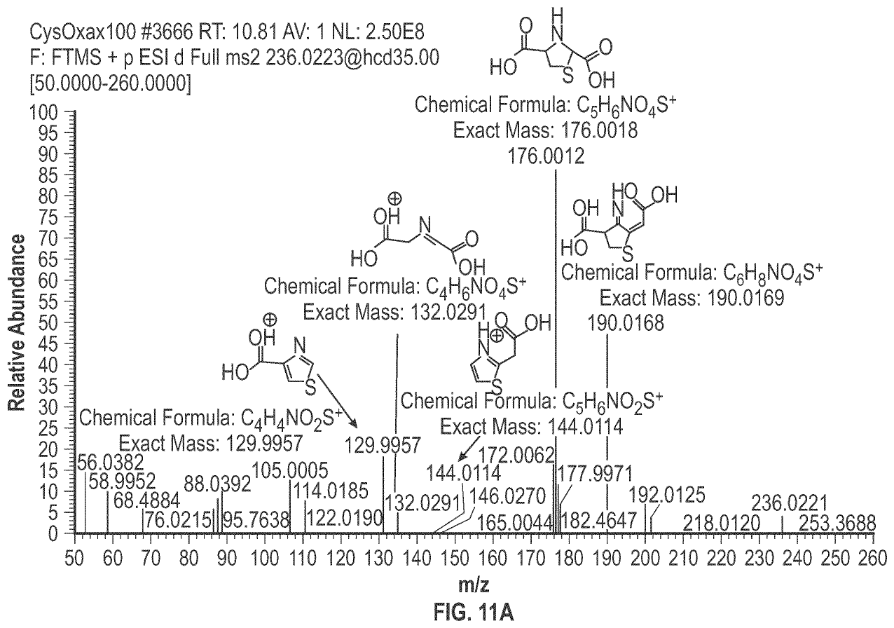

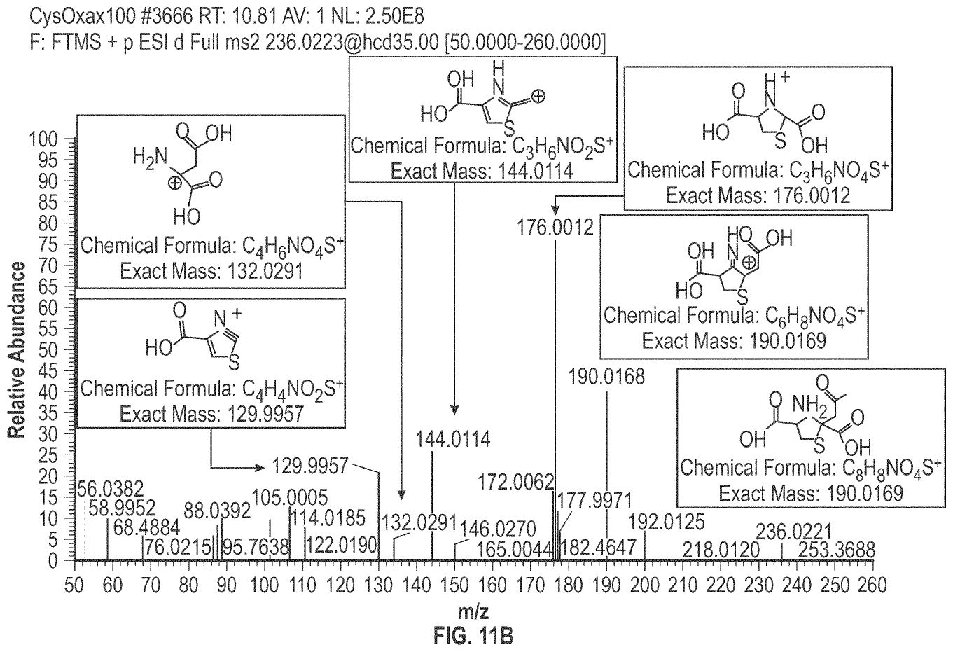

Furthermore, for Cys-AKG and Cys-Oxa, spectral features of 250.0376 (FIG. 8) and 236.0220 (FIG. 10) were found to be matched with the theoretical mass of C₈H₁₂NO₆S⁺ and C₇H₁₀NO₆S⁺, respectively. In a similar fashion as the analysis for Cys-Pyr, parent ions of 250.0376 at 11.18 min and 236.0220 at 9.20 min were fragmented into daughter ions shown in FIGS. 9A and 9B and FIGS. 11A and 11B, with predicted fragment structures applied to the tandem mass spectra shown therein. These results are consistent with the proposed chemical structures shown in FIG. 5, validating the formation of the complexes of interest.

Also solutions diluted 10 times were used to inject on LC-MS as described above to test the purity, the results show that the produced thiazolidine solutions (cysteine source solutions) were substantially pure for thiazolidines.

Note that we observed that in the Cys/Oxa solution, the complexes were a thiazolidine mixture of Cys-Pyr and Cys-Oxa. Without being bound by theory, the mixture of Cys-Pyr and Cys-Oxa is attributed to the following proposed chemical reaction of Cys-Oxa:

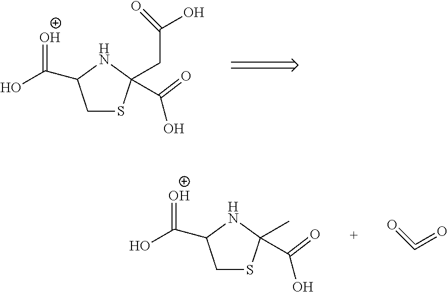

The complex with a mass of 236.0220 was used as the chemical marker to quantify the Cys-Oxa concentration in the cell culture samples.

NMR studies were also performed confirming the production of certain thiazolidines described herein (data now shown).

### Synthesized Thiazolidine Stability in Mammalian Cell Culture Feed

In-house feed was prepared with 20× concentrated thiazolidine solutions (cysteine source solutions; relative to the final desired concentration in the feed matrix). The finished feed solutions were aged at 4° C. for 10 days. As shown in Table 4, solutions exhibited negligible free thiol presence, indicating the stability of thiazolidines within the feed matrix, attesting to their reliability and longevity.

TABLE 4
DTNB Assay Detected Free Thiol in Cell Culture Feed Variants

| | | | Free thiol |
|---|---|---|---|
| Testing Group | Cell Culture Feed variants | | conc. (mM) |
| Positive Control | 45 mM Cys solution | | 45 |
| Negative Control | Feed Variant 1 | no Cys | 0.42 |
| Thiazolidine | Feed Variant 2 | Cys-Pyr | 0.25 |
| Thiazolidine | Feed Variant 3 | Cys-AKG | 0.12 |
| Thiazolidine | Feed Variant 4 | Cys-Oxa | 0.03 |
| Thiazolidine | Feed Variant 5 | 1/3Cys-PYR | 0.07 |
| | | 1/3Cys-AKG | |
| | | 1/3Cys-OXA | |

| | | |
|---|---|---|
| Blank | Tris Buffer | 0 |

### Example 4: Performance Evaluation of Synthesized Thiazolidine as Solo Cysteine Source for Cell Culture

Four diverse CHO cell lines (CL_1, CL_2, CL_3, and CL_4) producing either monoclonal antibodies or fusion proteins were used during fed-batch cell culturing in ambr15 to evaluate cell culture performance and product quality for feed media with solo Cys variants as different Cys delivery methods. The three synthesized thiazolidines options of Cys-Pyr, Cys-AKG, Cys-Oxa were compared with three other Cys delivery methods: Cys and n-acetyl-Cys mixture (Cys/NAC), di-lysyl-Cystine peptide (KCCK), and Cystine high-pH side solution.

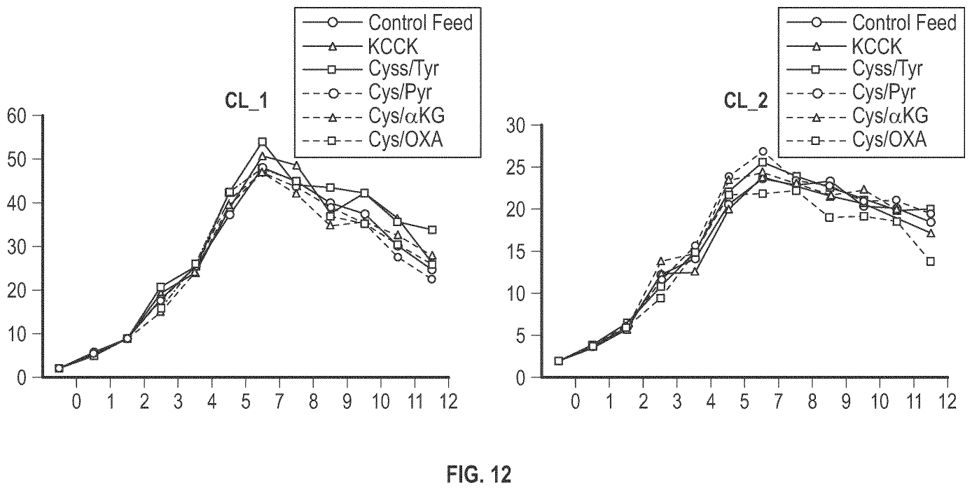

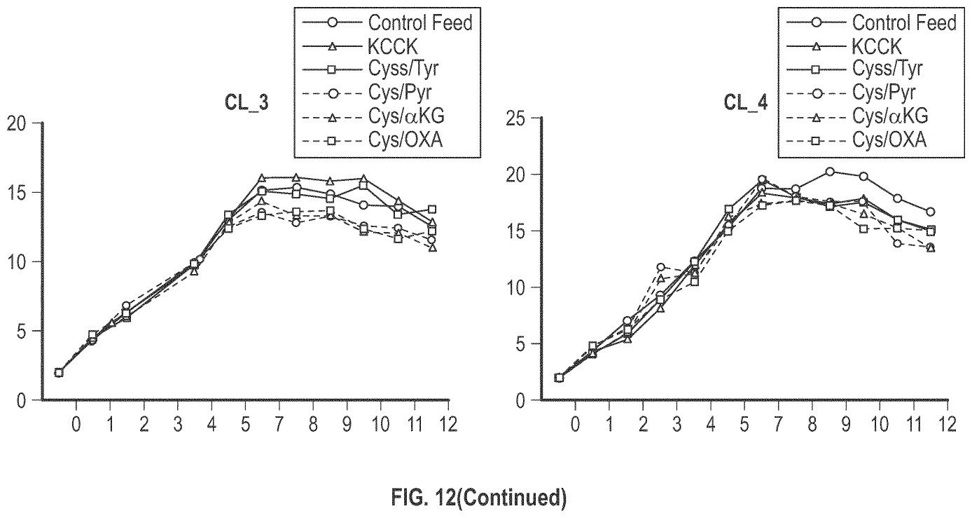

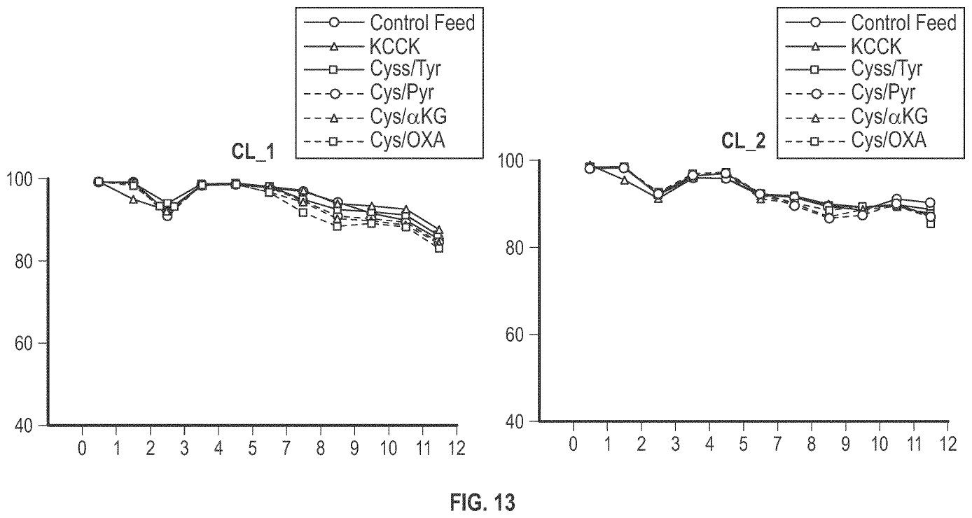

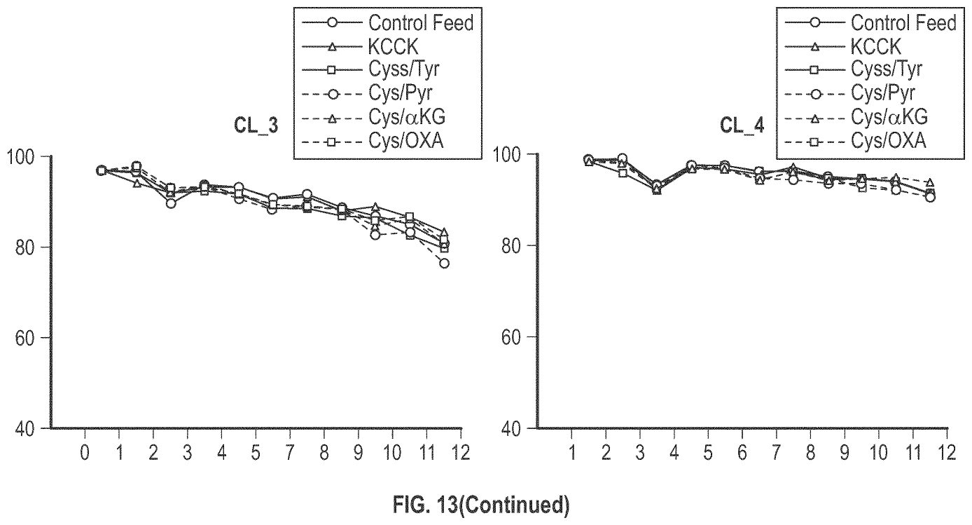

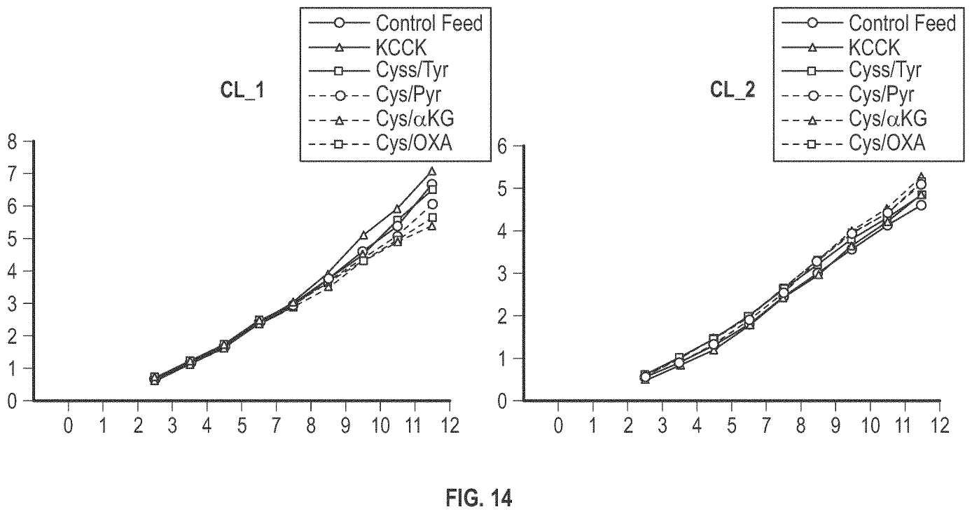

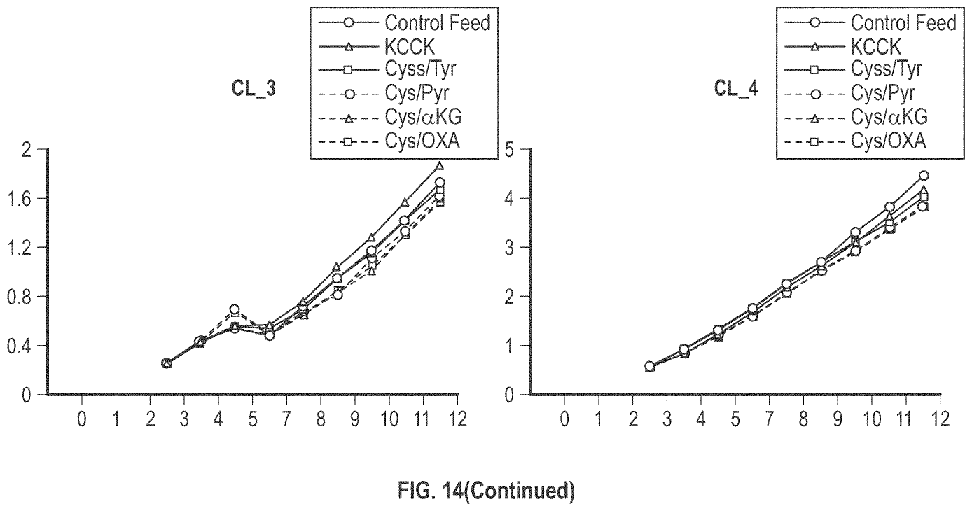

No obvious differences were found for using the three synthesized thiazolidines of Cys-Pyr, Cys-AKG, Cys-Oxa from other known Cys delivery method, regarding cell culture performance (FIGS. 12-14) and product quality attributes (not shown). Thus, the cysteine source solutions provided herein provide adequate nutrients for culturing cysteine-dependent cells while avoiding the known drawbacks of directly using cysteine to supplement cell culture media.

### Example 5: Metabolite Analysis of Synthesized Thiazolidine as Solo Cysteine Source for Cell Culture

Metabolite analysis by LC-MS was performed on daily spent media from intensified fed-batch processes in 3 L bioreactors. Specifically, the analysis was with three conditions: cell culture feed paired with a cystine side solution, cell culture feed containing Cys-Pyr thiazolidine, cell culture feed containing Cys-AKG thiazolidine in accordance with the Examples above, wherein the Cys-Pyr and Cys-AKG stock solutions were prepared according to the methods in the Examples above.

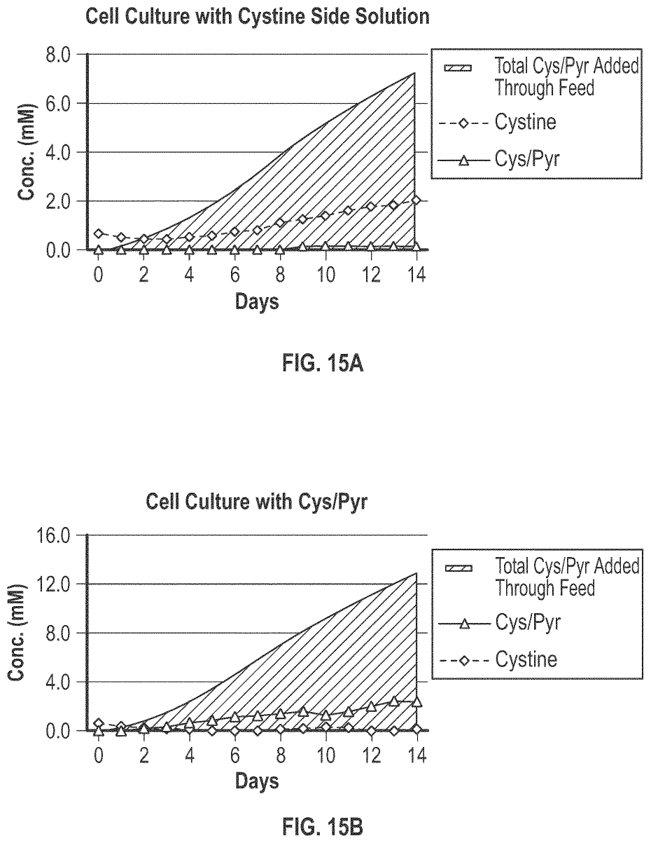

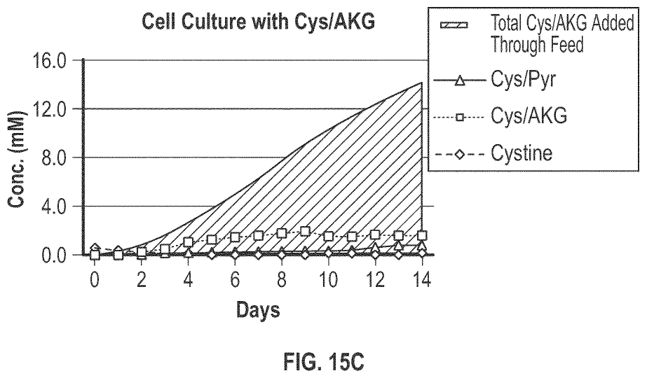

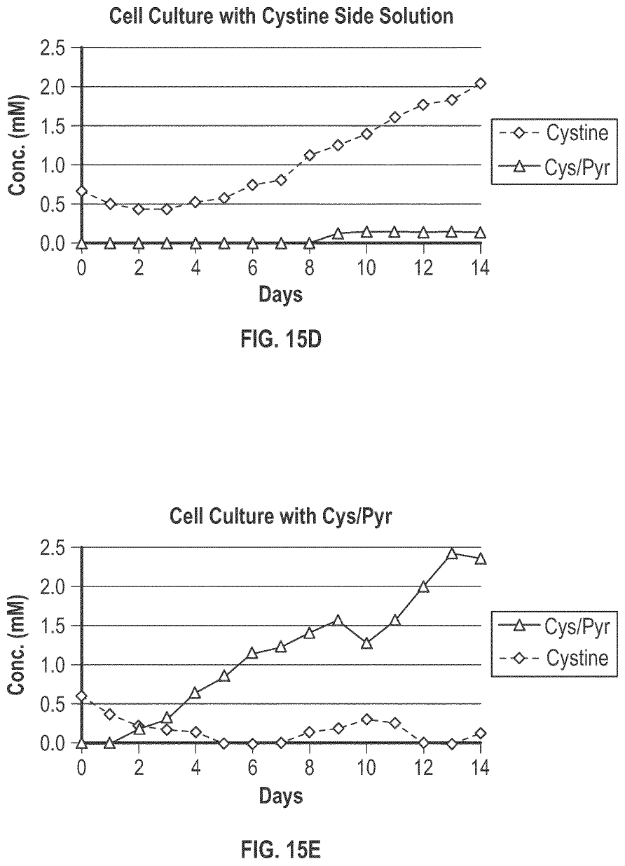

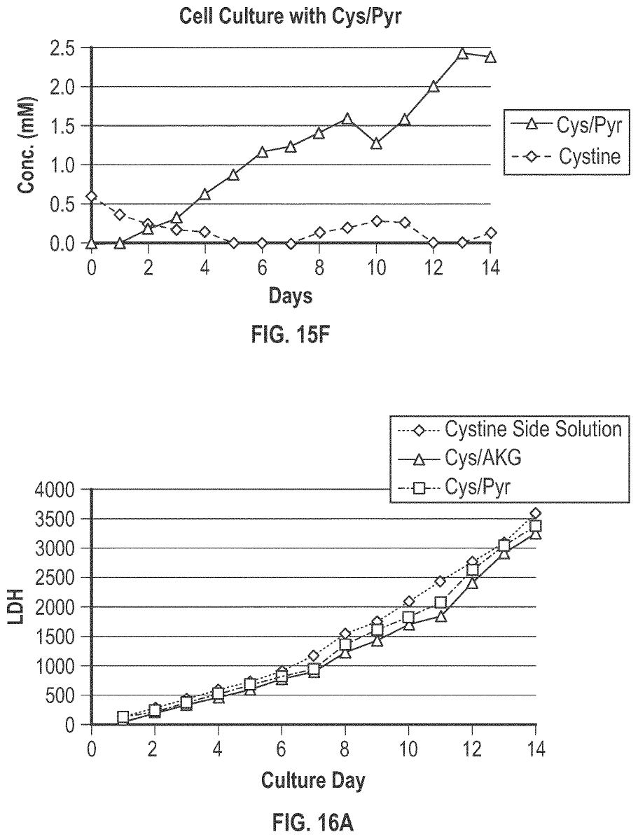

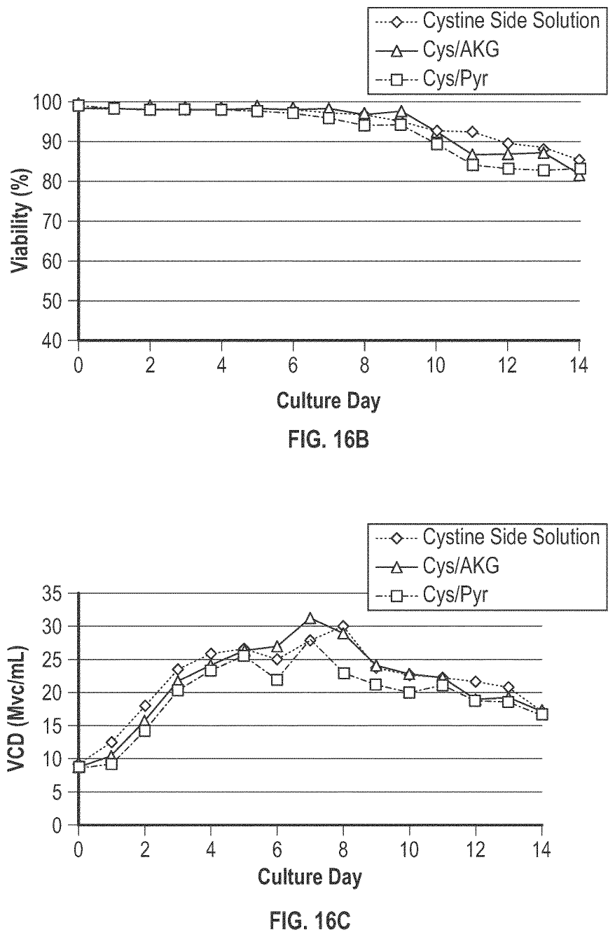

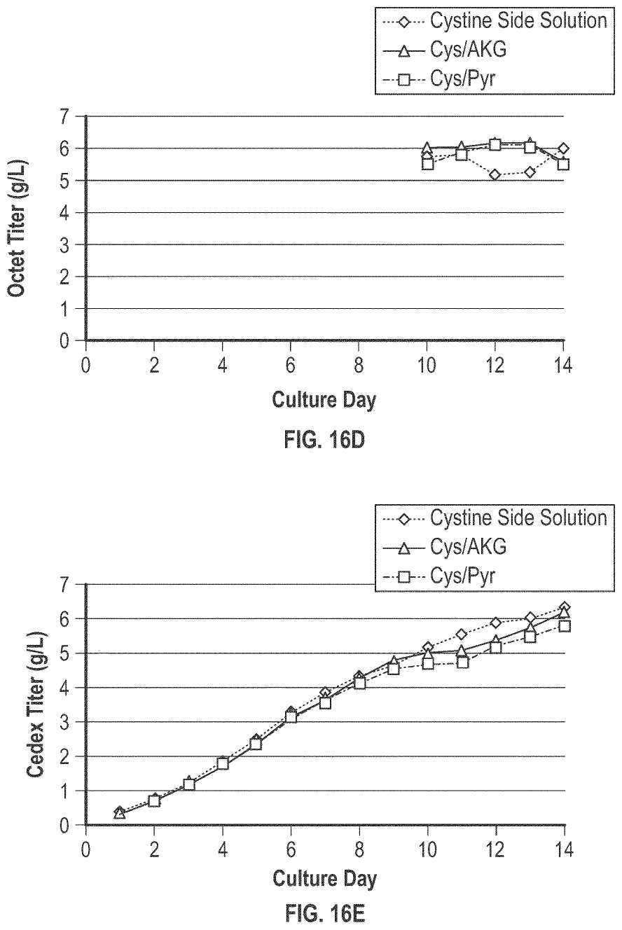

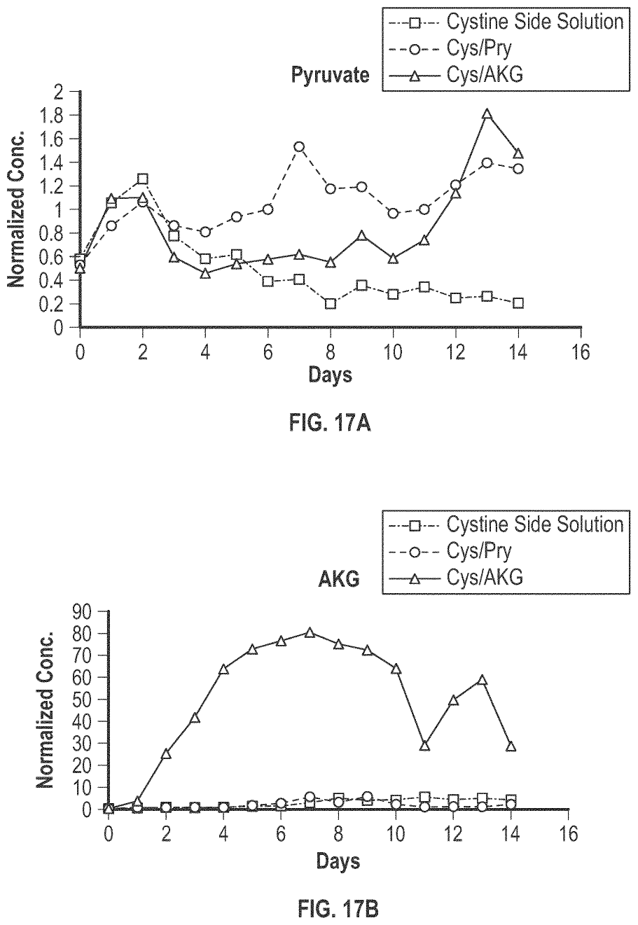

The normalized concentrations of cysteine source in daily spent media (free cysteine is converted cystine due oxidized measuring environment) are shown in FIGS. 15A-15C. FIGS. 15D-15E are provided to show zoomed in plots of certain data presented in FIGS. 15A-15C. As shown in FIGS. 15A-15C, the cells were able to consume thiazolidines as single cysteine source. As shown in FIGS. 17A-17B, measurements of free pyruvate and alpha-ketoglutarate indicate cells can convert the thiazolidines to native cysteine and the associated alpha-ketoacid. Comparable amounts of cysteine sources were consumed between the cystine side solution and thiazolidine conditions. Comparable cell culture performance was observed between tested conditions as assessed via LDH, viability, VCD, and titer (FIG. 16A-16E). Thus, these data demonstrate that the cysteine source solutions containing thiazolidines taught herein can be used as a single Cys source for the cell culture of cells capable of producing products, such as recombinant polypeptides, e.g., antibodies.

What is claimed is:

1. A method of making a cysteine source solution suitable for use with a cell culture, the method comprising:
reacting cysteine with an alpha-ketoacid compound of formula (A) 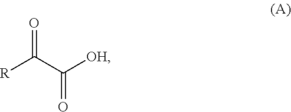
or the corresponding carboxylate anion, in an aqueous solution to produce the cysteine source solution comprising a thiazolidine compound of formula (B) 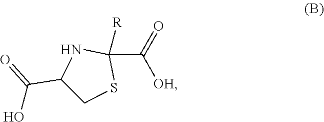
wherein R is optionally substituted C₁-C₆ alkyl,
wherein the reaction is performed with:
a concentration of the cysteine of at least about 30 mM, and/or
a concentration of the alpha-ketoacid compound of formula (A), or
corresponding carboxylate anion thereof, of at least about 30 mM, and/or
at a pH of about 1.5 to about 12.5, and/or
a ratio the cysteine and the alpha-ketoacid compound of formula (A) of about 2:1 to about 1:2.

2. The method of claim 1, wherein the reacting is not performed in a cell culture medium.

3. The method of claim 1 or 2, wherein the reacting is performed for 5 hours or less.

4. The method of any one of claims 1-3, wherein the residual concentration of cysteine or cystine in the cysteine source solution is about 0.5 mM or less.

5. The method of any one of claims 1-4, wherein the residual concentration of the alpha-ketoacid compound of formula (A) is about 0.5 mM or less.

6. The method of any one of claims 1-5, wherein the purity of the thiazolidine compound of formula (B) in the resulting cysteine source solution is at least about 95% of the original cysteine concentration.

7. The method of any one of claims 1-6, wherein the concentration of cysteine reacted with the alpha-ketoacid compound of formula (A) is about 30 mM to about 5000 mM.

8. The method of any one of claims 1-7, wherein the concentration of cysteine reacted with the alpha-ketoacid compound of formula (A) is at least about 150 mM.

9. The method of any one of claims 1-8, wherein the concentration of cysteine reacted with the alpha-ketoacid compound of formula (A) is at least about 1800 mM.

10. The method of any one of claims 1-9, wherein the alpha-ketoacid compound of formula (A) is pyruvic acid (Pyr) or pyruvate.

11. The method of any one of claims 1-9, wherein the alpha-ketoacid compound of formula (A) is alpha-ketoglutaric acid (AKG) or alpha-ketoglutarate.

12. The method of any one of claims 1-9, wherein the alpha-ketoacid compound of formula (A) is oxaloacetic acid (Oxa) or oxaloacetate.

13. The method of any one of claims 1-12, wherein the concentration of the alpha-ketoacid compound of formula (A) reacted with cysteine is about 30 mM to about 5000 mM.

14. The method of any one of claims 1-13, wherein the concentration of the alpha-ketoacid compound of formula (A) reacted with cysteine is at least about 150 mM.

15. The method of any one of claims 1-14, wherein the concentration of the alpha-ketoacid compound of formula (A) reacted with cysteine is at least about 1800 mM.

16. The method of any one of claims 1-15, wherein the reacting cysteine with the alpha-ketoacid compound of formula (A) is conducted at a pH from about 3 to about 12.

17. The method of any one of claims 1-16, wherein the reacting cysteine with the alpha-ketoacid compound of formula (A) is conducted at a pH of about 4.1.

18. The method of any one of claims 1-16, wherein the reacting cysteine with the alpha-ketoacid compound of formula (A) is conducted at a pH of about 7.

19. The method of any one of claims 1-16, wherein the reacting cysteine with the alpha-ketoacid compound of formula (A) is conducted at a pH of about 12.

20. The method of any one of claims 1-19, wherein the cysteine and alpha-ketoacid compound of formula (A) are reacted at a concentration ratio from 1:1 to 1:2 cysteine: alpha-ketoacid.

21. The method of any one of claims 1-20, wherein the cysteine and alpha-ketoacid compound of formula (A) are reacted at a concentration ratio of 1:1 cysteine: alpha-ketoacid.

22. The method of any one of claims 1-21, wherein cysteine is L-cysteine.

23. A method of making a cysteine source solution suitable for use with a cell culture, the method comprising:
reacting at least about 1800 mM cysteine with at least about 1800 mM of pyruvic acid, or the corresponding carboxylate anion, in an aqueous solution to produce the cysteine source solution comprising 2-methyl-1,3-thiazolidine-2,4-dicarboxylic acid,
wherein the reaction is performed starting at a pH of about 3.5 to about 12.

24. The method of claim 23, wherein the reaction is performed starting at a pH of about 4.1.

25. The method of claim 23, wherein the reaction is performed starting at a pH of about 7.

26. A method of making a cysteine source solution suitable for use with a cell culture, the method comprising:
reacting at least about 1800 mM cysteine with at least about 1800 mM of alpha-ketoglutaric acid, or the corresponding carboxylate anion, in an aqueous solution to produce the cysteine source solution comprising 2-(2-carboxyethyl) thiazolidine-2,4-dicarboxylic acid,
wherein the reaction is performed starting at a pH of about 4 to about 8.

27. The method of claim 26, wherein the reaction is performed at a pH of about 7.

28. A method of making a cysteine source solution suitable for use with a cell culture, the method comprising:
reacting at least about 1800 mM cysteine with at least about 1800 mM of oxaloacetic acid, or the corresponding carboxylate anion, in an aqueous solution to produce the cysteine source solution comprising 2-(carboxymethyl) thiazolidine-2,4-dicarboxylic acid,
wherein the reaction is performed starting at a pH of about 4 to about 8.

29. The method of claim 27, wherein the reaction is performed at a pH of about 7.

30. The method of any one of claims 23-29, wherein the reacting is performed for 2 hours or less.

31. The method of any one of claims 23-30, wherein the residual concentration of cysteine or cystine in the cysteine source solution is about 0.5 mM or less.

32. The method of any one of claims 23-31, wherein the residual concentration of pyruvic acid, alpha-ketoglutaric acid, or oxaloacetic acid, or the corresponding carboxylate anion of any, is about 0.5 mM or less.

33. The method of any one of claim 23-32, wherein the purity of the thiazolidine in the resulting cysteine source solution is at least about 95%.

34. A cysteine source solution produced according to the method of any one of claims 1-33.

35. A method of making a cell culture medium, the method comprising
admixing a basal cell culture medium with a cysteine source solution to make the cell culture medium, wherein the cysteine source solution is produced according to any one of claims 1-33.

36. The method of claim 35, wherein the cell culture medium is suitable for cysteine-dependent cells.

37. The method of claim 35 or 36, wherein the basal cell culture medium is not suitable for cysteine-dependent cells without further modification.

38. The method of any one of claims 35-37, wherein the basal cell culture medium has a pH of about 5 to about 8.

39. The method of any one of claims 35-38, further comprising preparing the thiazolidine side solution according to the methods of any one of claims 1-29.

40. The method of any one of claims 35-39, further comprising adjusting the pH of the basal cell culture medium.

41. The method of any one of claims 35-40, further comprising QS the cell culture medium after admixing the basal cell culture medium and the cysteine side solution.

42. The method of any one of claims 35-41, wherein the basal cell culture medium is substantially free of cysteine and cystine.

43. A method of culturing a cysteine-dependent cell, the method comprising culturing the cysteine-dependent cell in a cell culture medium made according to any one of claims 35-42.

44. A method of producing a polypeptide, the method comprising culturing a cysteine-dependent cell in a cell culture medium made according to any one of claims 35-42, and obtaining the polypeptide therefrom.

45. The method of claim 44, wherein the polypeptide is a therapeutic polypeptide or a precursor thereof.

46. The method of claim 45, wherein the therapeutic polypeptide or the precursor thereof is an antibody or a fragment thereof.

47. The method of claim 46, wherein the antibody or a fragment thereof is an antibody drug conjugate.

48. A cell culture system comprising:
a cell; and
a basal cell culture medium admixed with the cysteine source solution of claim 34, or a dried or semi-dried form thereof.

49. The cell culture system of claim 48, wherein the basal cell culture medium is substantially free of cysteine and cystine.

50. A method of culturing a cell, the method comprising
providing a cell culture medium to the cell,
wherein the cell culture medium comprises a basal culture medium and a cysteine source solution comprising a thiazolidine, and
wherein and the cell culture medium does not comprise more than about 20% cysteine relative to the amount of the thiazolidine in the cell culture medium; and
culturing the cell.

51. The method of claim 50, wherein the cell is a cysteine-dependent cell.

52. The method of claim 50 or 51, wherein the cell culture medium is not suitable for culturing the cell without further including cysteine or a source thereof.

53. The method of any one of claims 50-52, wherein the cysteine source solution is the sole source of cysteine for the cell.

54. The method of any one of claims 50-53, wherein the cell culture medium does not comprise cysteine.

55. The method of any one of claims 50-54, wherein the cysteine source solution is produced according to any one of the claims 1-33.

56. The method of any one of claims 50-55, wherein the cell culture comprises a fed-batch cell culture.

57. The method of claim 56, wherein the fed-batch cell culture is performed for a duration of 1 to 21 days.

58. The method of claim 57, wherein the fed-batch cell culture is performed for a duration of 14 days.

59. The method of any one of claims 56-58, wherein the cysteine source solution has a concentration of the thiazolidine of about 5 mM to about 50 mM.

60. The method of claim 59, wherein the cysteine source solution is added to the cell culture daily.

61. The method of claim 59 or 60, wherein the cysteine source solution is added throughout the fed-batch culture process.

62. The method of claim 60 or 61, wherein the volume of cysteine source solution added to the cell culture is based on the viable cell density.

63. The method of any one of claims 56-62, wherein the fed-batch cell culture is seeded at a cell density of about 1×10⁶ to about 8×10⁶ viable cells/mL.

64. The method of any one of claims 56-63, wherein the peak density of viable cells is 15×10⁶ to about 50×10⁶ cells/mL.

65. The method of any one of claims 56-64, wherein the cell culture has a viable cell density of about 15×10⁶ to about 50×10⁶ viable cells/mL.

66. The method of any one of claims 56-65, further comprising harvesting when the cell viability in the cell culture percentage drops to about 70% to about 80%.

67. The method of any one of claims 56-66, wherein the daily feeding volumes are about 0.5% to about 8.5% of the working volume per day.

68. The method of any one of claims 1-67, wherein the cell is a mammalian host cell that is transformed with a gene encoding a biomolecule of interest.

69. The method of claim 68, wherein the mammalian host cell is a CHO cell, a HeLa cell, a HEK293 cell, a Vero cell.

70. The method of claim 68, wherein the mammalian host cell is a CHO cell.

71. The method of any one of claims 68-70, wherein the biomolecule comprises an antibody or an antigen-binding fragment thereof, an enzyme, a viral vector comprising a therapeutic transgene, a gene editing complex, or a recombinant enzyme.

72. The method of claim 71, wherein the antibody or an antigen-binding fragment thereof comprises a monoclonal antibody (mAb), a bispecific antibody, a trispecific antibody, or an immunoglobulin single variable domain.

73. A concentrated basal cell culture medium comprising a thiazolidine.

74. The concentrated basal cell culture medium of claim 73, wherein the concentrated basal cell culture medium is 2× to 10×.

75. The concentrated basal cell culture medium of claim 73 or 74, wherein the basal cell culture medium is substantially free of cysteine or cystine.
